SEQUENCE LISTING (91 sequences)

SEQ ID NO: 1 - DNA, 30 bp, synthetic construct
  source 1..30: mol_type = other DNA
taaaagagatcgtgcgcccatataattaaa

SEQ ID NO: 2 - DNA, 30 bp, synthetic construct
  source 1..30: mol_type = other DNA
tatgttgttcgtcgagcatacccaaagtca

SEQ ID NO: 3 - DNA, 30 bp, synthetic construct
  source 1..30: mol_type = other DNA
acaaacttcccgcgttgtctgttgtacacg

SEQ ID NO: 4 - DNA, 30 bp, synthetic construct
  source 1..30: mol_type = other DNA
atggcccgacatgttgttgttctcccaaca

SEQ ID NO: 5 - DNA, 30 bp, synthetic construct
  source 1..30: mol_type = other DNA
gagagttgtttgttccaaggtgtcaaaggg

SEQ ID NO: 6 - DNA, 30 bp, synthetic construct
  source 1..30: mol_type = other DNA
cctcgctgtaactgttgtctccatcccaaa

SEQ ID NO: 7 - DNA, 30 bp, synthetic construct
  source 1..30: mol_type = other DNA
acacattccatgttgtccctactgtcaacg

SEQ ID NO: 8 - DNA, 30 bp, synthetic construct
  source 1..30: mol_type = other DNA
cgatcgtgttcaactgtgtgttcccaccaa

SEQ ID NO: 9 - DNA, 30 bp, synthetic construct
  source 1..30: mol_type = other DNA
agtgttgttgttataagtagacagcgaagg

SEQ ID NO: 10 - DNA, 30 bp, synthetic construct
  source 1..30: mol_type = other DNA
aacaggcttcactgttctttgtcccgccaa

SEQ ID NO: 11 - DNA, 30 bp, synthetic construct
  source 1..30: mol_type = other DNA
gaccattgttatgttgtatcacgcaacacc

SEQ ID NO: 12 - DNA, 30 bp, synthetic construct
  source 1..30: mol_type = other DNA
acgcgtgttctgctgtcttcctccaacaaa

SEQ ID NO: 13 - DNA, 30 bp, synthetic construct
  source 1..30: mol_type = other DNA
acaacagcgtgttgttgtgttaacccgtcg

SEQ ID NO: 14 - DNA, 30 bp, synthetic construct
  source 1..30: mol_type = other DNA
atcaacatgtatgtgtgttcccatccagcc

SEQ ID NO: 15 - DNA, 30 bp, synthetic construct
  source 1..30: mol_type = other DNA
acccgccttgtaacttgtcattgtaagcac

SEQ ID NO: 16 - DNA, 30 bp, synthetic construct
  source 1..30: mol_type = other DNA
caccttcgcaatcacttgtttcgtcccaca

SEQ ID NO: 17 - DNA, 30 bp, synthetic construct
  source 1..30: mol_type = other DNA
cgcaatgttgaaccgttgttcaccttcacg

SEQ ID NO: 18 - DNA, 30 bp, synthetic construct
  source 1..30: mol_type = other DNA
gcccgtttaacatgttctacttcccgcaca

SEQ ID NO: 19 - DNA, 30 bp, synthetic construct
  source 1..30: mol_type = other DNA
agccaccactgttgttgtaagctgttcgag

SEQ ID NO: 20 - DNA, 30 bp, synthetic construct
  source 1..30: mol_type = other DNA
cgacctgttcattcctatgttcccacccaa

SEQ ID NO: 21 - DNA, 30 bp, synthetic construct
  source 1..30: mol_type = other DNA
aacccaattgtcatgtgtcctttgccgaca

SEQ ID NO: 22 - DNA, 30 bp, synthetic construct
  source 1..30: mol_type = other DNA
cgcctaagtatgtcacttgtttcgcccgaa

SEQ ID NO: 23 - DNA, 30 bp, synthetic construct
  source 1..30: mol_type = other DNA
tttaattatatgggcgcacgatctctttta

SEQ ID NO: 24 - DNA, 30 bp, synthetic construct
  source 1..30: mol_type = other DNA
tgactttgggtatgctcgacgaacaacata

SEQ ID NO: 25 - DNA, 30 bp, synthetic construct
  source 1..30: mol_type = other DNA
cgtgtacaacagacaacgcgggaagtttgt

SEQ ID NO: 26 - DNA, 30 bp, synthetic construct
  source 1..30: mol_type = other DNA
tgttgggagaacaacaacatgtcgggccat

SEQ ID NO: 27 - DNA, 30 bp, synthetic construct
  source 1..30: mol_type = other DNA
ccctttgacaccttggaacaaacaactctc

SEQ ID NO: 28 - DNA, 30 bp, synthetic construct
  source 1..30: mol_type = other DNA
tttgggatggagacaacagttacagcgagg

SEQ ID NO: 29 - DNA, 30 bp, synthetic construct
  source 1..30: mol_type = other DNA
cgttgacagtagggacaacatggaatgtgt

SEQ ID NO: 30 - DNA, 30 bp, synthetic construct
  source 1..30: mol_type = other DNA
ttggtgggaacacacagttgaacacgatcg

SEQ ID NO: 31 - DNA, 30 bp, synthetic construct
  source 1..30: mol_type = other DNA
ccttcgctgtctacttataacaacaacact

SEQ ID NO: 32 - DNA, 30 bp, synthetic construct
  source 1..30: mol_type = other DNA
ttggcgggacaaagaacagtgaagcctgtt

SEQ ID NO: 33 - DNA, 30 bp, synthetic construct
  source 1..30: mol_type = other DNA
ggtgttgcgtgatacaacataacaatggtc

SEQ ID NO: 34 - DNA, 30 bp, synthetic construct
  source 1..30: mol_type = other DNA
tttgttggaggaagacagcagaacacgcgt

SEQ ID NO: 35 - DNA, 30 bp, synthetic construct
  source 1..30: mol_type = other DNA
cgacgggttaacacaacaacacgctgttgt

SEQ ID NO: 36 - DNA, 30 bp, synthetic construct
  source 1..30: mol_type = other DNA
ggctggatgggaacacacatacatgttgat

SEQ ID NO: 37 - DNA, 30 bp, synthetic construct
  source 1..30: mol_type = other DNA
gtgcttacaatgacaagttacaaggcgggt

SEQ ID NO: 38 - DNA, 30 bp, synthetic construct
  source 1..30: mol_type = other DNA
tgtgggacgaaacaagtgattgcgaaggtg

SEQ ID NO: 39 - DNA, 30 bp, synthetic construct
  source 1..30: mol_type = other DNA
cgtgaaggtgaacaacggttcaacattgcg

SEQ ID NO: 40 - DNA, 30 bp, synthetic construct
  source 1..30: mol_type = other DNA
tgtgcgggaagtagaacatgttaaacgggc

SEQ ID NO: 41 - DNA, 30 bp, synthetic construct
  source 1..30: mol_type = other DNA
ctcgaacagcttacaacaacagtggtggct

SEQ ID NO: 42 - DNA, 30 bp, synthetic construct
  source 1..30: mol_type = other DNA
ttgggtgggaacataggaatgaacaggtcg

SEQ ID NO: 43 - DNA, 30 bp, synthetic construct
  source 1..30: mol_type = other DNA
tgtcggcaaaggacacatgacaattgggtt

SEQ ID NO: 44 - DNA, 30 bp, synthetic construct
  source 1..30: mol_type = other DNA
ttcgggcgaaacaagtgacatacttaggcg

SEQ ID NO: 45 - DNA, 30 bp, synthetic construct
  source 1..30: mol_type = other DNA
tgatgatgtgtgatgatgtgtgatgatgtg

SEQ ID NO: 46 - DNA, 30 bp, synthetic construct
  source 1..30: mol_type = other DNA
catgtgatgtgtgtgatgatgtgtgatgat

SEQ ID NO: 47 - DNA, 30 bp, synthetic construct
  source 1..30: mol_type = other DNA
ctgtgatgtgtgatgatgatgtgtgatgat

SEQ ID NO: 48 - DNA, 30 bp, synthetic construct
  source 1..30: mol_type = other DNA
tgatgatgtgtgatgatgtgtgatgtgtga

SEQ ID NO: 49 - DNA, 30 bp, synthetic construct
  source 1..30: mol_type = other DNA
tgatgatgtgtgatgatgtgtgatgatgat

SEQ ID NO: 50 - DNA, 30 bp, synthetic construct
  source 1..30: mol_type = other DNA
gtgtgatgtgtgatgtgtgatgatgtgtga

SEQ ID NO: 51 - DNA, 30 bp, synthetic construct
  source 1..30: mol_type = other DNA
tgtgtgatgatgtgtgatgatgtgtgatga

SEQ ID NO: 52 - DNA, 30 bp, synthetic construct
  source 1..30: mol_type = other DNA
tgatgatgtgtgatgtgtgatgatgatgat

SEQ ID NO: 53 - DNA, 30 bp, synthetic construct
  source 1..30: mol_type = other DNA
gtgtgatgtgatgtgtgatgatgtgtgatg

SEQ ID NO: 54 - DNA, 30 bp, synthetic construct
  source 1..30: mol_type = other DNA
gatgtgtgatgtgtgatgtgtgatgatgtg

SEQ ID NO: 55 - DNA, 30 bp, synthetic construct
  source 1..30: mol_type = other DNA
tgatgatgtgtgatgtgtgatgatgtgatg

SEQ ID NO: 56 - DNA, 30 bp, synthetic construct
  source 1..30: mol_type = other DNA
gtgtgatgatgtgtgatgatgtgtgatgtg

SEQ ID NO: 57 - DNA, 30 bp, synthetic construct
  source 1..30: mol_type = other DNA
tgatgtgtgatgatgtgtgatgtgtgatga

SEQ ID NO: 58 - DNA, 30 bp, synthetic construct
  source 1..30: mol_type = other DNA
gtgtgatgtgtgatgatgtgtgatgatgtg

SEQ ID NO: 59 - DNA, 30 bp, synthetic construct
  source 1..30: mol_type = other DNA
tgatgatgatgtgtgatgatgtgtgatgtg

SEQ ID NO: 60 - DNA, 30 bp, synthetic construct
  source 1..30: mol_type = other DNA
tgatgatgtgtgatgtgtgatgatgatgtg

SEQ ID NO: 61 - DNA, 30 bp, synthetic construct
  source 1..30: mol_type = other DNA
tgcatgatgtgtgtgatgatgtgtgatgtg

SEQ ID NO: 62 - DNA, 30 bp, synthetic construct
  source 1..30: mol_type = other DNA
gatgtgtgtgatgtgatgtgtgatgatgtg

SEQ ID NO: 63 - DNA, 30 bp, synthetic construct
  source 1..30: mol_type = other DNA
atgatgtgtgatgtgtgatgatgtgtgatg

SEQ ID NO: 64 - DNA, 30 bp, synthetic construct
  source 1..30: mol_type = other DNA
tgatgatgtgtgatgtgatgtgtgatgatg

SEQ ID NO: 65 - DNA, 30 bp, synthetic construct
  source 1..30: mol_type = other DNA
tgatgatgtgtgatgatgatgatgtgtgat

SEQ ID NO: 66 - DNA, 30 bp, synthetic construct
  source 1..30: mol_type = other DNA
tgatgatgtgtgatgtgtgatgtgatgtga

SEQ ID NO: 67 - DNA, 30 bp, synthetic construct
  source 1..30: mol_type = other DNA
tgatgatgtgtgatgtgtgtgatgatgtga

SEQ ID NO: 68 - DNA, 30 bp, synthetic construct
  source 1..30: mol_type = other DNA
tgtgatactcagttgtatactgatgtgatg

SEQ ID NO: 69 - DNA, 30 bp, synthetic construct
  source 1..30: mol_type = other DNA
cttgaggatttgtgatgacatgtgatgttg

SEQ ID NO: 70 - DNA, 30 bp, synthetic construct
  source 1..30: mol_type = other DNA
tgtgatactggattctagtgtgatgtgatg

SEQ ID NO: 71 - DNA, 30 bp, synthetic construct
  source 1..30: mol_type = other DNA
tgatcactgaggtttatgtgtgatgtgatg

SEQ ID NO: 72 - DNA, 30 bp, synthetic construct
  source 1..30: mol_type = other DNA
tgatctgatgttggatgaatgtgtgatgtg

SEQ ID NO: 73 - DNA, 30 bp, synthetic construct
  source 1..30: mol_type = other DNA
tgttaatactgttggacatgtgtgatgatg

SEQ ID NO: 74 - DNA, 30 bp, synthetic construct
  source 1..30: mol_type = other DNA
ctcagcttgtgaatgacttgtgatgttgtg

SEQ ID NO: 75 - DNA, 30 bp, synthetic construct
  source 1..30: mol_type = other DNA
tgtctgaaccttggctgttatgtgatgatg

SEQ ID NO: 76 - DNA, 30 bp, synthetic construct
  source 1..30: mol_type = other DNA
tgtgaatttgtgagctctcctgatgtgatg

SEQ ID NO: 77 - DNA, 30 bp, synthetic construct
  source 1..30: mol_type = other DNA
tgaatatatttgatagtgatgtgatgatga

SEQ ID NO: 78 - DNA, 30 bp, synthetic construct
  source 1..30: mol_type = other DNA
tgttggttccacttggatgatgatgtgatg

SEQ ID NO: 79 - DNA, 30 bp, synthetic construct
  source 1..30: mol_type = other DNA
tgactcagttgatgaatttgtgatgtgtga

SEQ ID NO: 80 - DNA, 30 bp, synthetic construct
  source 1..30: mol_type = other DNA
tgatgatactgggagtttaatgtgatgttg

SEQ ID NO: 81 - DNA, 30 bp, synthetic construct
  source 1..30: mol_type = other DNA
tgtgattgaagtttgataacatgttgatga

SEQ ID NO: 82 - DNA, 30 bp, synthetic construct
  source 1..30: mol_type = other DNA
tgtcatgactgattgttgatgatgatgatg

SEQ ID NO: 83 - DNA, 30 bp, synthetic construct
  source 1..30: mol_type = other DNA
cttctgagtgtggagtttatgtgatgactg

SEQ ID NO: 84 - DNA, 30 bp, synthetic construct
  source 1..30: mol_type = other DNA
atgaactattagattgtgattgatgtgatg

SEQ ID NO: 85 - DNA, 30 bp, synthetic construct
  source 1..30: mol_type = other DNA
tgagattgtgcgatttgtctgagatgtgat

SEQ ID NO: 86 - DNA, 30 bp, synthetic construct
  source 1..30: mol_type = other DNA
tggatgaagttgtagatacttgtgatgttg

SEQ ID NO: 87 - DNA, 30 bp, synthetic construct
  source 1..30: mol_type = other DNA
tgtgatgtcatgtcatttggactgatgatg

SEQ ID NO: 88 - DNA, 30 bp, synthetic construct
  source 1..30: mol_type = other DNA
tgattggtttggaattgctgacgtgtgatg

SEQ ID NO: 89 - DNA, 30 bp, synthetic construct
  source 1..30: mol_type = other DNA
tgtgatttatgcctcatgaatgatgatgat

SEQ ID NO: 90 - DNA, 30 bp, synthetic construct
  source 1..30: mol_type = other DNA
ctctgtgattctgtgagccatgtgatgttg

SEQ ID NO: 91 - DNA, 30 bp, synthetic construct
  source 1..30: mol_type = other DNA
ttaaacctgatgtgtttcatgactgtgatg

(19) United States
(12) Patent Application Publication
(10) Pub. No.: US 2025/0111890 A1
(43) Pub. Date: Apr. 3, 2025

(54) APTAMER DESIGN BY REINFORCEMENT LEARNING BASED FINE-TUNING OF GENERATIVE LANGUAGE MODELS

(71) Applicant: X Development LLC, Mountain View, CA (US)

(72) Inventors: Jon DEATON, San Mateo, CA (US); Ryan POPLIN, Newark, CA (US); Ray NAGATANI, San Francisco, CA (US); Michelle WYNN, Carlsbad, CA (US); Anand PAI, San Francisco, CA (US); Joshua D'ARCY, Chicago, IL (US)

(73) Assignee: X Development LLC, Mountain View, CA (US)

(21) Appl. No.: 18/375,092

(22) Filed: Sep. 29, 2023

(51) Int. Cl. G16B 15/30; G06N 5/022; G16B 40/20

(52) U.S. Cl. CPC G16B 15/30; G06N 5/022; G16B 40/20

(57) ABSTRACT

The present disclosure relates to a closed loop aptamer development system that leverages in vitro experiments and in silico computation and artificial intelligence-based techniques to iteratively improve a process for identifying binders that can bind a molecular target. Particularly, aspects of the present disclosure are directed to obtaining, using an experimental assay, experimental data for a set of aptamers. The experimental data includes multiple pairs of data, each pair of data having: (i) an aptamer sequence for an aptamer from a set of aptamers, and (ii) a measurement for the characteristic of the aptamer with respect to a given target. A reward model is fine-tuned, using the experimental data, to predict a function-approximation metric for the characteristic of each aptamer in the set of aptamers. A decoder model is fine-tuned for generating novel aptamer sequences based on the function-approximation metric generated by the reward model for the novel aptamer sequences.

### SEQUENCE LISTING

The instant application contains a Sequence Listing which has been submitted electronically in XML format and is hereby incorporated by reference in its entirety. Said XML copy, created on Oct. 18, 2023, is named 105328-1379705_SL.xml and is 81,737 bytes in size.

### FIELD

The present disclosure relates to the development of aptamer sequences, and in particular to a closed loop aptamer development system that leverages in vitro experiments and in silico computation and artificial intelligence-based techniques to iteratively improve a process for identifying binders that can bind a molecular target.

### BACKGROUND

Aptamers are short sequences of single-stranded oligonucleotides (e.g., anything that is characterized as a nucleic acid, including xenobases). The sugar backbone of the single-stranded oligonucleotides functions as the acid and the A (adenine), T (thymine), C (cytosine), G (guanine) refers to the base. An aptamer can have modifications on either the acid or the base. Aptamers have been shown to selectively bind to specific targets (e.g., proteins, protein complexes, peptides, carbohydrates, inorganic molecules, organic molecules such as metabolites, cells, etc.) with high binding affinity, frequently in the picomolar to nanomolar range. Further, aptamers can be highly specific given they are designed to only interact with certain molecules, particular protein isoforms, or even a specific conformational state of their target protein. Thus, aptamers can be used to, for example, bind to disease-signature targets to facilitate a diagnostic process, bind to a treatment target to effectively deliver a treatment (e.g., a therapeutic or a cytotoxic agent linked to the aptamer), bind to target molecules within a mixture to facilitate purification, bind to a target to neutralize its biological effects, etc. However, the utility of an aptamer hinges on the degree to which it effectively binds to a target.

Frequently, an iterative experimental process (e.g., Systematic Evolution of Ligands by EXponential Enrichment (SELEX)) is used to identify aptamers that are selectively bound to target molecules with high affinity. In the iterative experimental process, a nucleic acid library of oligonucleotide strands (aptamers) are incubated with a target molecule. Then, the target-bound oligonucleotide strands are separated from the unbound strands and amplified via polymerase chain reaction (PCR) to seed a new pool of oligonucleotide strands. This selection process is continued for many rounds (e.g., 6-15) with increasingly stringent conditions, which ensure that the oligonucleotide strands obtained have the highest affinity to the target molecule.

The nucleic acid library typically includes 1014-1015 random oligonucleotide strands (aptamers). However, there are approximately a septillion (1024) different aptamers that could be considered, making it impractical to explore this full space of candidate aptamers. Moreover, given that present-day experiments can only test a fraction of the possible aptamers in the library, it is highly likely that optimal aptamer selection is not currently being achieved. Accordingly, while substantive studies on aptamers have progressed since the introduction of the SELEX process, it would take an enormous amount of resources and time to experimentally evaluate a septillion (1024) different aptamers every time a new target is proposed. In particular, there is a need for improving upon current experimental limitations with scalable artificial intelligence-based modeling techniques to identify aptamers and derivatives thereof that selectively bind to target molecules with high affinity.

### I. BRIEF SUMMARY

In some embodiments, a computer-implemented method is provided comprising obtaining, using an experimental assay, experimental data for a set of aptamers. The experimental data includes multiple pairs of data, each pair of data having: (i) an aptamer sequence for an aptamer from a set of aptamers, and (ii) a measurement for the characteristic of the aptamer with respect to a given target. A reward model is fine-tuned, using the experimental data, to predict a function-approximation metric for the characteristic of each aptamer in the set of aptamers. A decoder model is fine-tuned for generating novel aptamer sequences based on the function-approximation metric generated by the reward model for the novel aptamer sequences.

In some embodiments, a computer-implemented method is provided comprising fine-tuning a decoder model for generating novel aptamer sequences for a set of novel aptamers, where fine-tuning of the decoder model comprises inputting aptamer sequences for a set of aptamers into a decoder model, where each aptamer of the set of aptamers has a characteristic with respect to a given target that is measured at a desirable level, the decoder model comprises a function that is configured to return an action given a state, preceding nucleotides in an aptamer sequence are the state, and a nucleotide chosen to come next in the aptamer sequence is the action; generating, using the decoder model, novel aptamer sequences for a set of novel aptamers predicted to have a measurement at the desirable level for the characteristic of a novel aptamer with respect to the given target, where the novel aptamer sequence are generated by choosing each nucleotide or action one at a time from left-to-right, conditioning auto-regressively on the state or preceding nucleotides; inputting the novel aptamer sequences into a reward model; predicting, using the reward model, one or more measurement proxies for the characteristics of each novel aptamer represented by each of the novel aptamer sequences, where the function-approximation metric is the reward; optimizing, using reinforcement learning, model parameters of the decoder model that dictate sampling probabilities of the actions used to generate the novel aptamer sequences, where the optimizing comprises; calculating, using a first loss function, a first loss based on the sampling probabilities and differences in the reward predicted for each of the novel aptamer sequences, where the first loss function is configured to optimize sequential decision for choosing each nucleotide or action to maximize the reward; and adjusting the model parameters based on the first loss to finetune the decoder model for generating subsequent novel aptamer sequences for a subsequent set of novel aptamers having a more desirable measurement for the characteristic; and providing the fine-tuned decoder model.

In some embodiments, the method further comprises that the fine-tuning is performed iteratively until an average reward is equal to or greater than an average reward target threshold.

In some embodiments, the computer-implemented method further comprises calculating the average reward using the function-approximation metric predicted for the characteristic of each novel aptamer; when the average reward is less than the average reward target threshold, optimizing, using the reinforcement learning, the model parameters of the decoder model; and when the average reward is equal; to or greater than the average reward target threshold, providing the fine-tuned decoder model.

In some embodiments, the decoder model is a generative language model with a decoder-only transformer architecture.

In some embodiments, the generative language model is pretrained using a corpus of short non-coding RNA sequences.

In some embodiments, the optimizing further comprises sampling the actions, where the actions have corresponding sampling probabilities and differences in the reward predicted for each of the novel aptamer sequences are measurable and associable to the actions.

In some embodiments, the computer-implemented method further comprises calculating, using a second loss function, a second loss based on the sampling probabilities and differences in the reward predicted for each of the novel aptamer sequences, where the second loss function is configured to compute entropy of the next nucleotide or action prediction distributions output by the decoder model, summed over an entire aptamer sequence; and adjusting the model parameters based on the first loss and the second loss to finetune the decoder model for generating the subsequent novel aptamer sequences for the subsequent set of novel aptamers having a more desirable measurement for the characteristic.

In some embodiments, optimization further comprises calculating, using a third loss function, a third loss based on the sampling probabilities and differences in the reward predicted for each of the novel aptamer sequences, where the third loss function is configured to apply a penalty for divergence of a next nucleotide or action prediction from a distribution of the decoder model; and adjusting the model parameters based on the first loss, the second loss, and the third loss to finetune the decoder model for generating the subsequent novel aptamer sequences for the subsequent set of novel aptamers having a more desirable measurement for the characteristic.

In some embodiments, the first loss function is a proximal policy optimization objective function, the second loss function is a sequence entropy bonus objective function, and the third loss function is a Kullback-Leibner (KL) divergence objective function.

In some embodiments, the computer-implemented method further comprises obtaining, using an experimental assay, experimental data for a set of aptamers, where the experimental data comprises multiple pairs of data, each pair of data comprising: (i) an aptamer sequence for an aptamer from the set of aptamers, and (ii) a measurement for the characteristic of the aptamer with respect to the given target; and fine-tuning, using the experimental data, the reward model to predict the function-approximation metric for the characteristic of each aptamer in the set of aptamers.

In some embodiments, the experimental assay is a hydrogel particle display plate assay.

In some embodiments, the characteristic is binding affinity or binding specificity of the aptamer with respect to the given target.

In some embodiments, the set of aptamers comprises less than 1000 aptamers.

In some embodiments, the reward model is an ensemble of a Bidirectional Encoder Representations from Transformers (BERT) model and an Extreme Gradient Boosted (XGBoost) decision tree model.

In some embodiments, the fine-tuning of the reward model comprises fine-tuning, using the experimental data, both the BERT model and the XGBoost decision tree model to predicted via regression the function-approximation metric for the characteristic of each aptamer in the set of aptamers.

In some embodiments, the fine-tuning of the BERT model comprises calculating, using a first reward loss function, a first reward loss for predicting the function-approximation metric for the characteristic of an aptamer, where the first reward loss represents an error between the function-approximation metric and the measurement for the characteristic of the aptamer obtained from the experimental data; and updating model parameters of the BERT model based on the first reward loss.

In some embodiments, the fine-tuning of the XGBoost decision tree model comprises calculating, using a second reward loss function, a second reward loss for predicting the function-approximation metric for the characteristic of an aptamer, where the second reward loss represents an error between the function-approximation metric and the measurement for the characteristic of the aptamer obtained from the experimental data; and updating model parameters of the XGBoost decision tree model based on the second reward loss.

In some embodiments, the computer-implemented method further comprises prior to the fine-tuning of the reward model, pretraining, using a corpus of non-coding RNA sequences (ncRNAs) and a Masked Language Model (MLM) objective, the BERT model to predict missing nucleotides in an input ncRNA sequence based on context provided by surrounding nucleotides, where the pretraining comprises masking some of nucleotides in the input ncRNA sequence to generate the missing nucleotides and training the model to predict the masked nucleotides based on the context of non-masked words.

In some embodiments, each pair of data further comprises: (i) the aptamer sequence for the aptamer from the set of aptamers, and (ii) a measurement for a characteristic of the aptamer and a different measurement for a different characteristic of the aptamer, and the method further comprise fine-tuning, using the experimental data, another reward model to predict a different function-approximation metric for the different characteristic of each aptamer in the set of aptamers.

In some embodiments, a computer-implemented method comprises obtaining initial sequence data for aptamers of an initial non-coding RNA database that bind to a target, do not bind to the target, or a combination thereof; identifying, by a reward machine learning model, a first set of aptamer sequences as satisfying one or more constraints, where the reward machine learning model comprises pre-trained model parameters learned from the initial sequence data, and the first set of aptamer sequences are derived from a subset of sequences from the initial sequence data, sequences from the first set of aptamer sequences are different from sequences from the initial sequence data; obtaining, using an in vitro binding selection process, subsequent sequence data for aptamers of a subsequent aptamer library that bind to the target, do not bind to the target, or a combination thereof, where the subsequent aptamer library comprises aptamers synthesized from the first set of aptamer sequences; identifying, by a generative language model with a decoder-only transformer architecture, a second set of aptamer sequences as satisfying the one or more constraints, where the second machine learning model is a generative decoder language model, where the second machine learning model comprises model parameters learned from the subsequent sequence data, and the second set of aptamer sequences are derived from a subset of sequences from the subsequent sequence data, sequences from a pool of sequences different from sequences from the subsequent sequence data, or a combination thereof; determining, using one or more in vitro assays, experimental data for aptamers synthesized from the second set of aptamer sequences; identifying a final set of aptamer sequences from the second set of aptamer sequences that satisfy the one or more constraints based on the experimental data associated with each aptamer; and outputting the final set of aptamer sequences.

In some embodiments, the computer-implemented method further comprises synthesizing one or more aptamers using the final set of aptamer sequences; and synthesizing a biologic using the one or more aptamers.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure will be better understood in view of the following non-limiting figures, in which:

FIG. 1 shows a diagram of how the three main components: laboratory measurements, reward model, and decoder model interact in an iterative process to design satisfactory candidate aptamer sequences in accordance with various embodiments.

FIG. 2 shows a data collection and modeling system for developing aptamers in accordance with various embodiments.

FIG. 3 shows a block diagram of workflow for training a reward model in accordance with various embodiments.

FIGS. 4A and 4B show masked base prediction accuracy and loss over the course of parameter update steps in pre-training of a reward model in accordance with various embodiments.

FIGS. 5A and 5B show scatter plots show training set (left) and test set (right) scatter plots of actual fluorescence value (x-axis) and model predicted fluorescence (y-axis) in accordance with various embodiments.

FIG. 6 shows a block diagram of workflow for training a decoder model in accordance with various embodiments.

FIGS. 7A and 7B show masked base prediction accuracy and loss over the course of parameter update steps in pre-training of a decoder model in accordance with various embodiments.

FIG. 8A shows a total number of unique sequences generated by the decoder in a pool of 64k generated sequences with (orange) and without (blue) the Sequence Entropy Bonus loss in accordance with various embodiments.

FIG. 8B shows top-scoring generated (SEQ ID NOS 45-91, respectively, in order of appearance by columns) from decoder fine-tuned with RLEF before (left) and after (right) the addition of the KL loss which penalizes divergence from the pre-trained distribution of non-coding RNAs as learned by the decoder in accordance with various embodiments.

FIG. 8C shows RLEF training run metrics over the course of 500 generation/update steps for fine-tuning a 12 layer decoder generative model for S1 RBD/Benzyl TNA sequences in accordance with various embodiments.

FIG. 8D shows reward model scores for random sequences (orange) in comparison with sequences generated from the RLEF fine-tuned decoder-only generative model (blue) in accordance with various embodiments.

FIGS. 9A and 9B show statistical tests comparing RLEF tuned decoder (blue) and decoder-ablation (blue) against random baseline in accordance with various embodiments.

FIG. 10 shows a comparison between RLEF generated sequences vs randomly searching for high-scoring sequences in accordance with various embodiments.

FIG. 11A shows the highest fluorescence/affinity sequences are generated via the RLEF method (highest purple dots) in accordance with various embodiments.

FIG. 11B shows the disclosed approach finds high affinity (e.g. ≥9 corrected fluorescence) on the 9T modality with significantly higher success rate as compared with the previous art of XGBoost classifier+random search in accordance with various embodiments.

FIG. 12 shows a block diagram of a pipeline for strategically identifying and generating binders of molecular targets in accordance with various embodiments.

FIG. 13 shows an exemplary workflow for training a reward and decoder model in accordance with various embodiments.

FIGS. 14A and 14B show an exemplary flow for aptamer development in accordance with various embodiments.

FIG. 15 shows an exemplary computing device in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

### INTRODUCTION

Identification of aptamers with ideal characteristics such as strong affinity and/or high specificity for target molecules such as proteins has the potential to greatly improve the field of drug discovery given that two of its major shortcomings include insufficient validation of therapeutic targets and insufficient specificity of drug candidates. However, given the extensive number of potential aptamer sequences (e.g., 1024 potential sequences) and the comparatively low throughput of methodologies to assess the binding affinity of candidates (e.g., dozens to thousands per week), finding optimal aptamers is highly unlikely. While in vitro selection based approaches (e.g., SELEX and display plate assays) can identify aptamers from libraries comprising of millions to trillions of candidates, there are several weaknesses with these approaches: (i) output candidate aptamers are ambiguous—it is challenging to know whether relatively strong binders in the library will actually be strong binders under physiological conditions; (ii) data is noisy-binding is dependent on every candidate encountering the target protein with the same relative frequency and variance from this can lead to many false negatives and some false positives; and (iii) experimental capacity is much smaller than the total search space (e.g., SELEX can test a maximum of ˜1014 candidates out of the 1024 aptamers in the library) making experimental validation of candidate aptamers unpredictable, expensive, and time consuming.

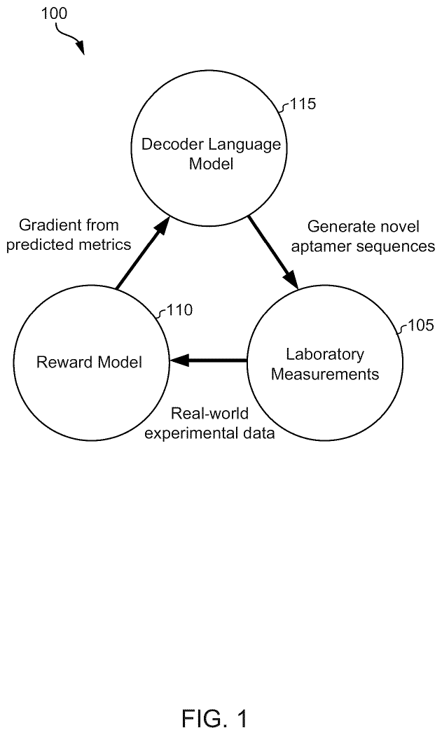

To address these challenges and others, an artificial intelligence-based solution is disclosed herein for fine-tuning a generative language model to design a diverse set of XNA aptamer sequences that satisfy a given characteristic such as high-predicted-affinity and/or high-predicted-specificity. FIG. 1 shows a conceptual diagram 100 of the interactions between the three components of the artificial intelligence-based solution. Fundamentally, experimental data from laboratory measurements 105 is used to construct a reward model 110, which is a machine learning model of a fitness landscape of aptamer sequences with respect to a given characteristic such as affinity to the target protein. The reward model 110 communicates information about this fitness landscape to a decoder machine learning language model 115 as the decoder learns how to design good candidate aptamer sequences (those that optimize for the given characteristic). Finally, sequences designed by the decoder machine learning language model 115 are synthesized in vitro to generate aptamers or oligonucleotides strands and tested experimentally (e.g., using a hydrogel particle display (PD) plate-based assay), to complete the loop. As should be understood to those of ordinary skill in the art, the result of the in vitro synthesis is physical aptamers or oligonucleotide strands not in silico or simulated aptamers or oligonucleotides strands.

In one exemplary aspect of the artificial intelligence-based solution, a computer-implemented method is disclosed that comprises: fine-tuning a decoder model for generating novel aptamer sequences for a set of novel aptamers, where fine-tuning of the decoder model comprises: inputting aptamer sequences for a set of aptamers into a decoder model, where: each aptamer of the set of aptamers has a characteristic with respect to a given target that is measured at a desirable level, the decoder model comprises a function that is configured to return an action given a state, preceding nucleotides in an aptamer sequence are the state, and a nucleotide chosen to come next in the aptamer sequence is the action; generating, using the decoder model, novel aptamer sequences for a set of novel aptamers predicted to have a measurement at the desirable level for the characteristic of a novel aptamer with respect to the given target, where the novel aptamer sequence are generated by choosing each nucleotide or action one at a time from left-to-right, conditioning auto-regressively on the state or preceding nucleotides; inputting the novel aptamer sequences into a reward model; predicting, using the reward model, a function-approximation metric for the characteristic of each novel aptamer represented by each of the novel aptamer sequences, where the function-approximation metric is the reward; optimizing, using reinforcement learning, model parameters of the decoder model that dictate sampling probabilities of the actions used to generate the novel aptamer sequences, where the optimizing comprises; calculating, using a first loss function, a first loss based on the sampling probabilities and differences in the reward predicted for each of the novel aptamer sequences, where the first loss function is configured to optimize sequential decision for choosing each nucleotide or action to maximize the reward; and adjusting the model parameters based on the first loss to finetune the decoder model for generating subsequent novel aptamer sequences for a subsequent set of novel aptamers having a more desirable measurement for the characteristic; and providing the fine-tuned decoder model.

This artificial intelligence-based solution is a significant adaptation of Reinforcement Learning from Human (RLHF), which is used to align large generative language models (e.g., GPT) to respond to prompts as a dialog agent. In the present instance, the artificial intelligence-based solution can be thought of more accurately as Reinforcement Learning from Experimental Feedback (RLEF) because human preferences are not used, rather experimental data are used from a laboratory assay, in a design-test loop. The key differences between RLHF and RLEF include the following without limitation.

In RLHF, reward models for reinforcement learning use a Decoder/Generative Pre-trained Transformer (GPT) model; whereas in the RLEF the reward model is a Bidirectional Encoder Representations from Transformers (BERT) model or an ensemble of a BERT model and an Extreme Gradient Boosted (XGBoost) decision tree model. BERT is bidirectional and thus considers both left and right context when making predictions. This makes it better suited for aptamer sequence analysis tasks such as binding affinity prediction where understanding the full context of the sequence is essential. Additionally, RLEF utilizes a transfer learning approach where prior to fine-tuning the reward and decoder models are pre-trained with self-supervised/unsupervised learning on a much more difficult set of starting data, i.e., a large corpus of short naturally occurring non-coding RNA sequences (ncRNAs) as described in further detail herein. Because ncRNAs display numerous aptamer-like characteristics (e.g., known target interaction, secondary structure, chemical modifications, etc.), the models are reinforced to (i) predict sequences with high binding affinities, (ii) generate a diverse pool of candidate sequences, and (iii) prioritize synthesizable sequences versus un-synthesizable sequences. Further, there is no supervised fine-tuning pre-step of the decoder model in RLEF because there is generally not enough data to actually fine tune the decoder model using such a supervised fine-tuning pre-step. Instead, the pre-training of the decoder model is relied on to model the distribution of biologically plausible non-coding RNA sequences as a “baseline model”.

Another important aspect of this artificial intelligence-based solution is that unlike RLHF that use binary “yes/no” feedback from a human, the RLEF is pre-trained on small amounts of continuous valued measurements from the laboratory (e.g., experimentally obtained data). In so doing, this approach does not optimize an outcome based on human feedback, instead it optimizes functional characteristics of aptamers measured from an experimental assay. Lastly, the key components of the RLEF: experimental assays, a reward model, and a decoder model are implemented in an iterative loop so that the decoder model is continuously generating its own sample pool and each iteration designs a more robust pool of candidate aptamers.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent. As used herein, when an action is “based on” something, this means the action is based at least in part on at least a part of the something.

It will be appreciated that techniques disclosed herein can be applied to assess other biological material (e.g., other binders such as monoclonal antibodies) rather than aptamers. For example, alternatively or additionally, the techniques described herein may be used to assess the interaction between any type of biologic material (e.g., a whole or part of an organism such as E. coli, or a biologic product that is produced from living organisms, contain components of living organisms, or derived from human, animal, or microorganisms by using biotechnology) and a target, and derive another type of biologic material therefrom based on the assessment.

### Data Collection and Modeling System

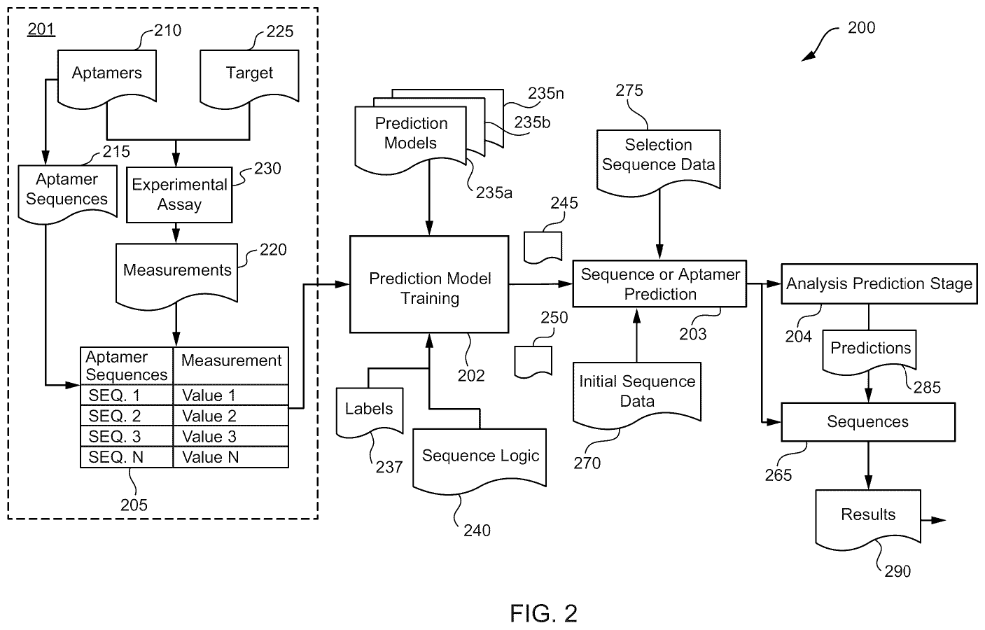

FIG. 2 shows a block diagram of a data collection and modeling system 200 for collecting experimental data for aptamers (e.g., a library of aptamers), training machine learning models using the experimental data for aptamers, and deploying the machine learning models to predict novel aptamer sequences. As shown in FIG. 2, the processes performed by the data collection and modeling system 200 in this example include several subsystem or service: a data collection subsystem 201, a prediction model training subsystem 202, a sequence or aptamer prediction subsystem 203, and an optional analysis prediction subsystem 204.

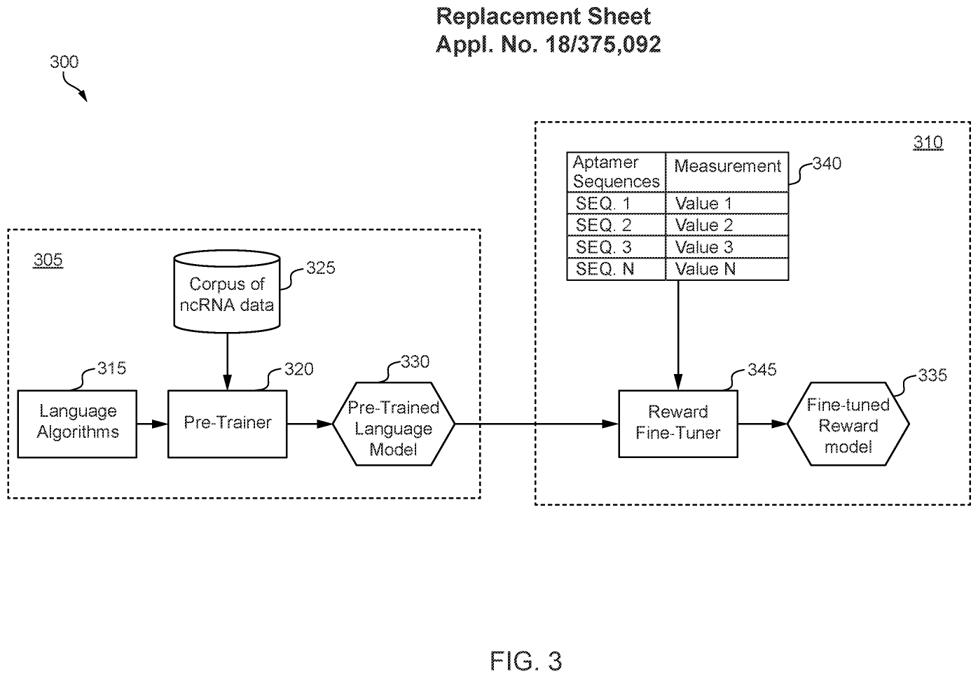

The data collection subsystem 201 facilitates the collection of experimental data 205. Experimental data 205 includes multiple pairs of data, where each pair of data comprises: (i) an aptamer sequence 215 for each of the aptamers 210, and (ii) a function-approximation metric 220 (e.g., fluorescence) for a characteristic (e.g., binding affinity) of each of the aptamer 210 with respect to a given target 225. The experimental data is collected for the purpose of training and fine-tuning a reward model to predict a function-approximation metric (also described herein as a measurement proxy) for the characteristic of each of the aptamers 210 (described in more detail in FIG. 3). For example, in the instance the characteristic is binding affinity, the aptamer sequence 215 is the input and the measurement 220 for binding affinity of the aptamer with respect to a given target is the ground truth label, which are used to train a supervised machine learning model to take as input a novel aptamer sequence and predict that aptamer's binding affinity to the given target.

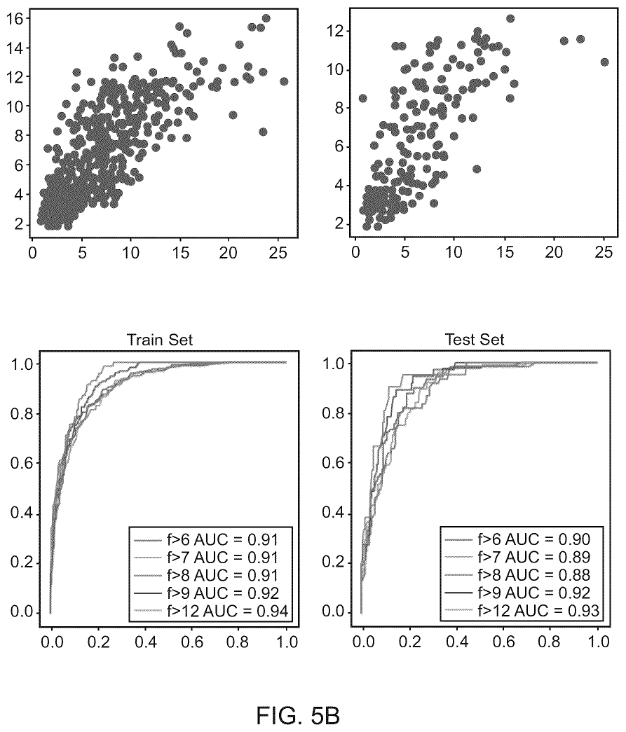

The workflow 200 includes running an in vitro experimental assay 230 on the aptamers 210 and the target 225 to obtain the measurement 220 for a characteristic (e.g., binding affinity) of each of the aptamers 210 with respect to the target 225. The aptamers 210 and target 225 (short sequences of single stranded oligonucleotides) may be obtained via one or more processes including those disclosed with respect the pipeline for strategically identifying and generating binders of molecular targets, as described in greater detail herein with respect to FIG. 5. For example, the set of aptamers 210 may be identified via in vitro bind selections (e.g., phage display or SELEX) that can bind to the target 225. The target 225 may be identified as a result of a query posed by a user (e.g., a client or customer). As used herein, the terms “a query” and “a given problem” are defined as a request from a user or an inquiry corresponding to an interaction between a target (e.g., a protein) and a molecule such as an aptamer. The terms are used interchangeably. The query or the given problem can include finding one or more aptamers that can bind or inhibit a target. The query or the given problem can include a particular number of aptamers to be found that can bind or inhibit a target. The query or the given problem can include particular interaction characteristics between the aptamer and the target according to the nature of the query or the problem. For example, a user may pose a query concerning identification of ten aptamers with the highest binding affinity for a given target or twenty aptamers with the greatest ability to inhibit activity of a given target. Accordingly, a solution to the given problem and a response to an inquiry are also used interchangeably. The response can include information corresponding to the target, the aptamer(s) found to bind or not bind the target (e.g., sequence data), the interaction characteristics, or the number of aptamers in the response or solution.

Experimental assay 230 is any in vitro assay (e.g., spectroscopic assays such as affinity selection mass spectrometry (ASMS), isothermal titration calorimetry (ITC), or optical biosensors such as surface plasmon resonance (SPR), biolayer interferometry (BLI), and grating-coupled interferometry (GCI), hydrogel assays, radioligand binding assays, and the like) that can be used to obtain a measurement for a characteristic (e.g., binding affinity) of an aptamer with respect to the given target 225. For example, if the characteristic of interest is binding affinity, then a hydrogel particle display plate assay can be used that measures the binding affinities of aptamers 210 relative to a target 215 one at a time in a multi-well (e.g., 96-well) plate by measuring fluorescence The fluorescence signal indicates an aptamer-target complex, and thus the greater the signal the higher the binding affinity.

As used herein, the term “binding affinity” means the free energy differences between native binding and unbound states, which measures the stability of native binding states (e.g., a measure of the strength of attraction between an aptamer and a target). As used herein, a “high binding affinity” is a result from stronger intermolecular forces between an aptamer and a target leading to a longer residence time at the binding site (higher “on” rate, lower “off” rate). The factors that lead to high affinity binding include a good fit between surface of the molecules in their ground state and charge complementary (i.e., stronger intermolecular forces between the aptamer and the target). These same factors generally also provide a high binding specificity for the targets, which can be used to simplify screening approaches aimed at developing strong therapeutic candidates that can bind the given molecular target. As used herein, the term “binding specificity” means the affinity of binding to one target relative to the other targets. As used herein, the term “high binding specificity” means the affinity of binding to one target is stronger relative to the other targets. Various aspects described herein design and validate aptamers as strong therapeutic candidates that can bind the given molecular target based on binding affinity. However, it should be understood that design and validation of aptamers could involve the assessment of characteristic(s) other than binding affinity and/or binding specificity.

The data collection subsystem 201 facilitates sequencing the aptamers 210 to obtain aptamer sequences 215 (i.e., a library of sequence reads associated with the aptamers 205). In some embodiments, the aptamers 210 are amplified (e.g., amplified by a PCR-based method). For example, a sequencing method may comprise amplification of a nucleic acid library. A nucleic acid library can be amplified prior to or after immobilization on a solid support (e.g., a solid support in a flow cell). Nucleic acid amplification includes the process of amplifying or increasing the numbers of a nucleic acid template and/or of a complement thereof that are present (e.g., in a nucleic acid library such as the aptamers 210), by producing one or more copies of the template and/or its complement. Amplification can be carried out by a suitable method. A nucleic acid library can be amplified by a thermocycling method or by an isothermal amplification method. In some embodiments a rolling circle amplification method is used. In some embodiments amplification takes place on a solid support (e.g., within a flow cell) where a nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support. In some embodiments, modified nucleic acid (e.g., nucleic acid modified by addition of adapters) is amplified.

Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). MPS sequencing sometimes makes use of sequencing by synthesis and certain imaging processes.

Sequencing by synthesis generally is performed by iteratively adding (e.g., by covalent addition) a nucleotide to a primer or preexisting nucleic acid strand in a template directed manner. Each iterative addition of a nucleotide is detected and the process is repeated multiple times until a sequence of a nucleic acid strand is obtained. The length of a sequence obtained depends, in part, on the number of addition and detection steps that are performed. In some embodiments of sequencing by synthesis, one, two, three or more nucleotides of the same type (e.g., A, G, C or T) are added and detected in a round of nucleotide addition. Nucleotides can be added by any suitable method (e.g., enzymatically or chemically). For example, in some embodiments a polymerase or a ligase adds a nucleotide to a primer or to a preexisting nucleic acid strand in a template directed manner. In some embodiments of sequencing by synthesis, different types of nucleotides, nucleotide analogues and/or identifiers are used. In some embodiments reversible terminators and/or removable (e.g., cleavable) identifiers are used. In some embodiments fluorescent labeled nucleotides and/or nucleotide analogues are used. In certain embodiments sequencing by synthesis comprises a cleavage (e.g., cleavage and removal of an identifier) and/or a washing step. In some embodiments the addition of one or more nucleotides is detected by a suitable method described herein or known in the art, non-limiting examples of which include any suitable imaging apparatus, a suitable camera, a digital camera, a CCD (Charge Couple Device) based imaging apparatus (e.g., a CCD camera), a CMOS (Complementary Metal Oxide Silicon) based imaging apparatus (e.g., a CMOS camera), a photo diode (e.g., a photomultiplier tube), electron microscopy, a field-effect transistor (e.g., a DNA field-effect transistor), an ISFET ion sensor (e.g., a CHEMFET sensor), the like or combinations thereof.

Any suitable MPS method, system or technology platform for conducting methods described herein can be used to obtain nucleic acid sequence reads. Non-limiting examples of MPS platforms include Illumina/Solex/HiSeq (e.g., Illumina's Genome Analyzer; Genome Analyzer II; HISEQ 2000; HISEQ), SOLID, Roche/454, PACBIO and/or SMRT, Helicos True Single Molecule Sequencing, Ion Torrent and Ion semiconductor-based sequencing (e.g., as developed by Life Technologies), WildFire, 5500, 5500xl W and/or 5500xl W Genetic Analyzer based technologies (e.g., as developed and sold by Life Technologies); Polony sequencing, Pyrosequencing, Massively Parallel Signature Sequencing (MPSS), RNA polymerase (RNAP) sequencing, LaserGen systems and methods, Nanopore-based platforms, chemical-sensitive field effect transistor (CHEMFET) array, electron microscopy-based sequencing (e.g., as developed by ZS Genetics, Halcyon Molecular), nanoball sequencing, the like or combinations thereof. Other sequencing methods that may be used to conduct methods herein include digital PCR, sequencing by hybridization, nanopore sequencing, chromosome-specific sequencing (e.g., using DANSR (digital analysis of selected regions) technology.

In some instances, complete or substantially complete aptamer sequences 215 are obtained via sequencing. In other instances, partial aptamer sequences 215 are obtained are obtained via sequencing. Nucleic acid sequencing generally produces a collection of sequence reads. As used herein, “reads” (e.g., “a read,” “a sequence read”) are nucleotide sequences produced by any sequencing process described herein or known in the art. Reads can be generated from one end of nucleic acid fragments (“single-end reads”), and sometimes are generated from both ends of nucleic acid fragments (e.g., paired-end reads, double-end reads). The length of a sequence read is often associated with the particular sequencing technology. The high-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments sequence reads are of a mean, median, average or absolute length of about 500 bp or more.

The aptamer sequences 215 are then combined with the measurements 220 to generate the experimental data 205. The experimental data 205 can be formatted into a data table. The data table can be any data structure that stores information in memory of a computing system, can be retrieved later, and updated as needed. As illustrated in FIG. 2, in some instances, the data table comprises columns, rows and cells that contain specific values for the aptamer sequences 215 and the measurements 220 (e.g., the fluorescent signal). The data table maintains the values for the aptamer sequences 215 and the measurements 220 in association with one another such that any system (e.g., a model training subsystem) accessing or running operations using the experimental data 205 is aware of or can determine those associations. The resulting experimental data 205 for aptamers 210 may be formed of data from multiple aptamers and experiments containing ˜hundreds to thousands of data set pairs (e.g., sequences, fluorescence).

The prediction model training subsystem 202 builds and trains one or more models 225a-225n (‘n’ represents any natural number) to be used by the other stages (which may be referred to herein individually as a model 225 or collectively as the models 225). For example, the models 225 can include one or more different type of models for generating sequences of aptamers not experimentally determined by a selection process but identified or designed (e.g., by a computational model) based on aptamers experimentally determined by a selection process. The models 225 may be used in the pipeline 100 described with respect FIG. 1 for identifying or designing high affinity binders for a given target. The models 225 can also include a model for predicting binding counts for the predicted sequences for derived aptamers. The models 225 can also include a model for predicting analytics such as binding affinity for the predicted sequences for derived aptamers. Still other types of prediction models may be implemented in other examples according to this disclosure.

A model 235 can be a machine learning model, such as a neural network, a convolutional neural network (“CNN”), e.g. an inception neural network, a residual neural network (“ResNet”) or NASNet provided by GOOGLE LLC from MOUNTAIN VIEW, CALIFORNIA, or a recurrent neural network, e.g., long short-term memory (“LSTM”) models or gated recurrent units (“GRUs”) models, or transformers such as a Bidirectional Encoder Representations from Transformers (BERT) model. A model 235 can also be any other suitable machine learning model trained to predict predicted sequences for derived aptamers, sequence counts or analytics for aptamer sequences, such as a support vector machine, decision tree (e.g., an Extreme Gradient Boosted (XGBoost) decision tree model), a three-dimensional CNN (“3DCNN”), regression model, linear regression model, ridge regression model, logistic regression model, a dynamic time warping (“DTW”) technique, a hidden Markov model (“HMM”), etc., or combinations of one or more of such techniques—e.g., CNN-HMM or MCNN (Multi-Scale Convolutional Neural Network). The data collection and machine learning modeling system 200 may employ one or more of same type of model or different types of models for aptamer sequence prediction, aptamer count prediction, and/or analysis prediction.

To train the various models 235 in this example, training samples for each model 235 are obtained or generated. The training samples for a specific model 235 can include the experimental data 205 for aptamers 210 as described with respect to data collection subsystem 201 and optional labels 237 corresponding to the experimental data 205. For example, for a model 235 to be utilized to identify or design an aptamer sequence, the input can be the aptamer sequence itself or features extracted from the sequence data associated with the aptamer sequence and optional labels 237 can include a binding-approximation metric, functional-approximation metric, and/or calculated fitness scores (a measure of how well each aptamer sequences satisfies one or more constraints) for the aptamer sequences. Similarly, for a model 235 to be utilized to predict a count or binding affinity for an aptamer sequence, the input can include the sequence and count features extracted from the initial sequence data and/or the sequence data associated with the sequence, and the optional labels 237 can include features indicating parameters for the count, binding affinity, or functional analysis (e.g., a binding approximation metric and/or functional-approximation metric) or a vector indicating probabilities for the count, binding affinity, functional analysis of the sequence data.

In some instances, the training process includes iterative operations to find a set of parameters for the model 235 that maximizes or minimizes an objective function (e.g., regression or classification loss) for the models 235. Each iteration can involve finding a set of parameters for the model 235 so that the value of the objective function using the set of parameters is smaller or greater than the value of the objective function using another set of parameters in a previous iteration. The objective function can be constructed to measure the difference between the outputs predicted using the models 235 and the optional labels 237 contained in the training samples. Once the set of parameters are identified, the model 235 has been trained and can be tested, validated, and/or utilized for prediction as designed.

In addition to the training samples, other auxiliary information can also be employed to refine the training process of the models 235. For example, sequence logic 240 can be incorporated into the prediction model training stage 205 to ensure that the sequences or aptamers, counts, and analysis predicted by a model 235 do not violate the sequence logic 240. For example, binding affinity (the strength of the binding interaction between an aptamer and a target) is a characteristic that can drive aptamers to be present in greater numbers in a pool of aptamer-target complexes after a cycle of selection process. This relationship can be expressed in the sequence logic 240 such that as the binding affinity variable increases the predictive count increases (to represent this characteristic), as the binding affinity variable decreases the predictive count decreases. Moreover, an aptamer sequence generally has inherent logic among the different nucleotides. For example, GC content for an aptamer is typically not greater than 60%. This inherent logical relationship between GC content and aptamer sequences can be exploited to facilitate the aptamer sequence prediction.

According to some aspects of the disclosure presented herein, the logical relationship between the binding affinity and count can be formulated as one or more constraints to the optimization problem for training the models 235. A training loss function that penalizes the violation of the constraints can be built so that the training can take into account the binding affinity and count constraints. Alternatively, or additionally, structures, such as a directed graph, that describe the current features and the temporal dependencies of the prediction output can be used to adjust or refine the features and predictions of the models 235. In an example implementation, features may be extracted from the initial sequence data and combined with features from the selection sequence data as indicated in the directed graph. Features generated in this way can inherently incorporate the temporal, and thus the logical, relationship between the initial library and subsequent pools of aptamer sequences after cycles of the selection process. Accordingly, the models 235 trained using these features can capture the logical relationships between sequence characteristics, selection cycles, aptamer sequences, and nucleotides.

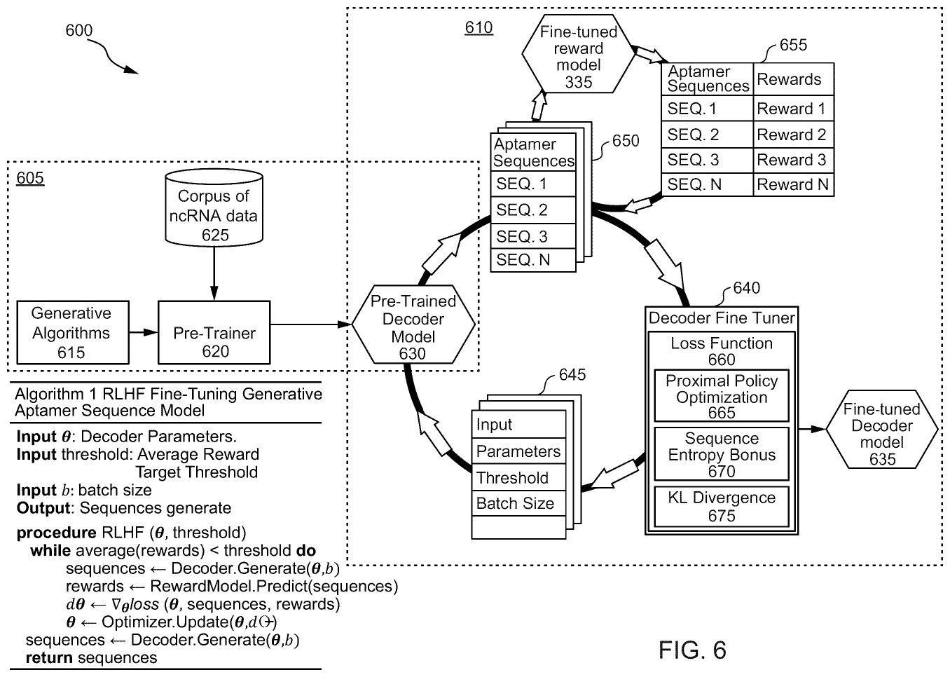

Although the training mechanisms described herein mainly focus on training a model 235, these training mechanisms can also be utilized to fine tune existing models 235 trained from other datasets as described in detail with respect to FIGS. 3 and 6. For example, in some cases, a model 235 might have been pre-trained using pre-existing aptamer sequence libraries or related RNA sequence libraries as described in detail with respect to FIGS. 3 and 6. In those cases, the models 235 can be retrained using the training samples containing initial sequence data, experimentally derived selection sequence data, and other auxiliary information as discussed herein.

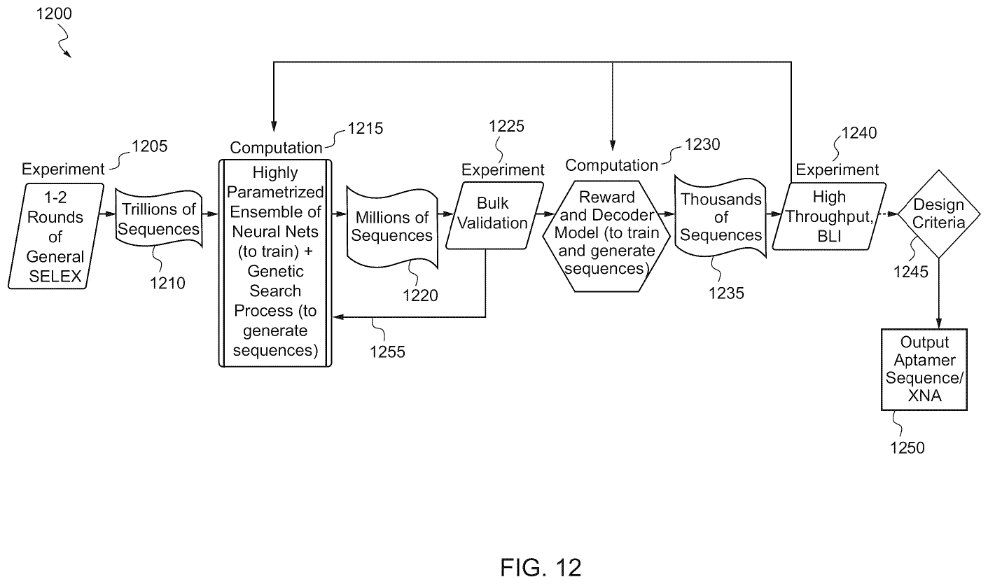

The prediction model training stage 205 outputs trained models 235 including trained nonlinear or highly parametrized models 245, trained language models or generative language models 250 (e.g., reward and decoder models describe in detail herein with respect to FIGS. 3-11B, optionally trained count prediction models, and optionally trained analysis prediction models. The trained nonlinear or highly parametrized models 245 and trained language models or generative language models 250 may be used in the sequence prediction subsystem 203 to identify or design sequences 265 based on a subset or all of the initial sequence data 270 (e.g., random sequence data), the selection sequence data 275 identified during the experimental selection process (e.g., blocks 1205-1240 described with respect to FIG. 12), or a combination thereof. The trained count prediction models may be used in the optional analysis prediction subsystem 204 to generate count predictions 285 for the identified sequences based on the initial sequence data 270 and/or the selection sequence data 275 identified during the experimental selection process (e.g., blocks 1205, 1225, and 1240 described with respect to FIG. 12). The trained analysis prediction models may be used in the optional analysis prediction subsystem 204 to generate analysis predictions 285 (e.g., a binary classifier such as binds to target or does not bind to target) for the identified sequences based on the initial sequence data 270 and/or the selection sequence data 275 identified during the experimental selection process (e.g., blocks 1205, 1225, and 1240 described with respect to FIG. 12). In some instances, the identified or designed sequences 265, count predictions 285, analysis predictions 285, or any combination thereof may be provided as results 290.

The results 290 may be used to synthesize aptamers to be validate or improved experimentally. The results 290 may be a solution to a given problem or a query (e.g., posed by a user). For example, in response to a query for top hundred aptamers that bind a given target, the results 290 may include the identity of sequences for a hundred aptamers with the highest count or binding affinity for the given target. As described with respect to FIG. 1, the results 290 may then be used to synthesize the aptamers to be used in low-throughput assays for characterizing or validating the results 290 as potential therapeutic candidates.

### Reward Model Training

FIG. 3 shows a block diagram of a workflow 300 that uses the experimental data 205 collected with respect to data collection subsystem 201 to train a supervised machine learning model such as a language model (LM). The machine learning model is trained to take as input an aptamer sequence and predict a function-approximation metric (e.g., fluorescence) for a characteristic (e.g., binding affinity) of an aptamer associated with the aptamer sequence. The training approach is to pre-train the machine learning model using a large corpus of short non-coding RNA sequences so that the machine learning model learns transferable knowledge from these biologically/molecular related sequences in a pre-training phase 305. Thereafter, the machine learning model is fine-tuned to predict a function-approximation metric for a characteristic (e.g., binding affinity with a given target) via supervised learning in a fine training phase 310.

In some embodiments, the machine learning model is a Bidirectional Encoder Representations from Transformers (BERT) regression based model. A BERT model is an open-source machine learning framework for language processing that helps to decipher ambiguous language in text by simultaneously gathering the context of a word from both the left and right direction. Transformers are a deep learning model in which each output element is connected to an input element, and the weights between them are dynamically calculated based upon their connection. At a basic level, transformers utilize an encoder-decoder method where the encoder reads the input, and the decoder produces the predicted value. However, because BERT models are designed to only generate language, (e.g., measurement for the characteristic of the aptamer), they do not require a decoder component. Further, BERT replicates the encoder architecture of the transformer model by layering multiple transformer encoders one over the other. In some instances, the BERT model comprises 12 or 24 layers in the encoder stack.

In other embodiments, the machine learning model is an ensemble of a BERT regression based model and an Extreme Gradient Boosted (XGBoost) decision tree regression based model. A XGBoost Regression Model is a type of supervised machine learning that utilizes ensembles of various machine learning algorithms (e.g., decision trees, linear regression, neural networks, nearest neighbor, naive bayes, and the like) to “boost” the attributes that led to misclassification of a previous algorithm such as a decision tree. In other words, the XGBoost approach addresses measurements that the model incorrectly predicted and gives them more weight during the next iteration of training. The ensemble learning of the XGBoost approach facilitates regularized boosting and prevents overfitting of the model.

Still other types and variants of machine learning model may be implemented in other embodiments according to this disclosure such as an XGBoost classifier based model, XGBoost regression based model, a BERT classifier based model, and the like. In any instance, this machine learning model is called herein the “Reward Model” (RM) since its output is later used in workflow 500 described with respect to FIG. 6 as the “reward” to train a different machine learning model (decoder model) in a Reinforcement Learning (RL) optimization algorithm.

One of the greatest challenges with training machine learning models such as LM models to predict a function-approximation metric for a characteristic (e.g., binding affinity) of an aptamer and design or predict candidate sequences capable of imparting such a characteristic to an aptamer is the limited availability of data. It is typical to have only a few hundred measurements of an XNA characteristic such as aptamer affinities to a given target. Moreover, the relationship between aptamer sequence and a characteristic such as protein target affinity is extraordinarily complex: the aptamer folds into a three-dimensional structure dictated by self-complementary Watson-Crick pairing. The three-dimensional structure of the aptamer defines availability of electrostatic interactions with the protein, which in turn determines the favorability of its interaction with the protein target. It is infeasible to robustly learn the details of this complex interaction “from scratch” from only a few hundred (or even thousands) of sequence/characteristic measurements.

Because of this, a transfer learning approach is utilized where prior to fine-tuning, language algorithms 315 (e.g., an encoder, regression, classifier, decision tree, etc. algorithm) are pre-trained in pre-training phase 305. The pre-training phase 305 comprises the pretrainer 320 inputting a large corpus 325 into the language algorithms 315, the pretrainer 320 trains the language algorithms 315 on the large corpus 325 using a supervised learning approach, and the pretrainer 320 outputs pre-trained language model(s) 330. The pretrainer 320 is part of a machine learning operationalization framework (e.g., the prediction model training subsystem 202 described with respect to FIG. 2) comprising hardware such as one or more processors (e.g., a CPU, GPU, TPU, FPGA, the like, or any combination thereof), memory, and storage that operates software or computer program instructions (e.g., TensorFlow, PyTorch, Keras, and the like) to execute arithmetic, logic, input and output commands for training the language algorithms 315. The large corpus 325 comprises examples of short naturally occurring non-coding RNA sequences (ncRNAs). ncRNAs are short oligonucleotides with structural similarities to XNA aptamers, and which also often perform aptamer-like functions like binding to proteins to affect their biological function. There are large, open datasets of non-coding RNA sequences generally available on the public databases, including RNACentral (˜30 million sequences), DASHR (˜80k sequences), and mirBase (˜100k sequences). By pre-training the language algorithms 315 via supervised learning on large datasets of non-coding RNAs, the language algorithms 315 learn transferable knowledge from ncRNA sequences such as patterns in the distribution of naturally occurring short single-stranded nucleobase molecules which serve a biological like protein binding. For example, how a sequence must be constituted in order to fold into a reliable secondary structure, or what features of a sequence enable it to interact with proteins.

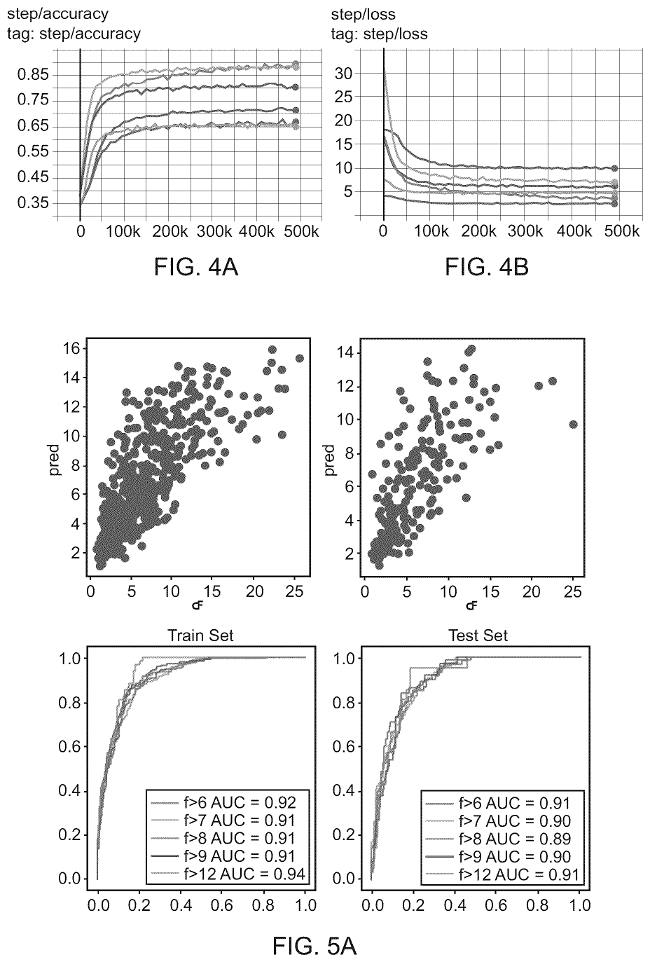

The pre-training of the language algorithms 315 is executed by the pretrainer 320 using a pre-training scheme such as Masked Language Modeling (MLM), Causal Language Modeling (CLM), or next sentence prediction (NSP). In some instances, the pre-training scheme is MLM. Under the MLM approach, a certain % of nucleobases in a given RNA sequence (e.g., 15%) from the large corpus 325 are randomly masked out and the language algorithms 315 are expected to predict those masked nucleobases based on context provided by surrounding nucleobases in that RNA sequence. Such a training scheme makes this model bidirectional in nature because the representation of the masked nucleobases is learnt based on the nucleobases that occur to its left as well as right. FIGS. 4A and 4B show pre-training of an exemplary 12-layer BERT model optimized for masked nucleobase prediction (MLM) on datasets of non-coding RNAs. FIG. 4A shows masked base prediction accuracy and FIG. 4B shows loss over the course of 500,000 parameter update steps in pre-training. The curves show light blue: RNAcentral, orange: DASHR, red: miRBase mature, miRBase hairpin, pink: training, green: validation.

After having been pre-trained, the pre-trained language model(s) 330 are ready to be fine-tuned using the actual experimental data from assays testing for an XNA aptamer characteristic. This approach is also referred to as “transfer learning” where accuracy on a task (in this case XNA aptamer/characteristic) is improved by first having learned a different but related task (e.g., masked nucleobase prediction (MLM) on datasets of non-coding RNAs). Fine-tuning the pre-trained language model(s) 330 to generate fine-tuned reward model(s) 335 (Reward Models) is a useful step in RLEF because it ultimately determines the fitness landscape which the decoder model will be tuned to optimize in workflow 500 described with respect to FIG. 5. For this reason, the quality of the reward model is a substantial determinant of the quality of the overall system.

The fine-tuning phase 310 comprises inputting experimental data 340 into a reward fine tuner 345, the fine tuner 345 fine-tuning the pre-trained language model(s) 330 using a supervised learning approach to predict a function-approximation metric for the characteristic of an aptamer (e.g., the fluorescence of a XNA aptamer indicative of binding affinity when the XNA aptamer is tested on a hydrogel PD plate-based assay), and outputs fine-tuned reward model(s) 335. The reward fine tuner 345 is also part of the machine learning operationalization framework (e.g., the prediction model training subsystem 202 described with respect to FIG. 2) comprising hardware such as one or more processors (e.g., a CPU, GPU, TPU, FPGA, the like, or any combination thereof), memory, and storage that operates software or computer program instructions (e.g., TensorFlow, PyTorch, Keras, and the like) to execute arithmetic, logic, input and output commands for fine-tuning the pre-trained language model(s) 330. The experimental data 340 comprises aptamer sequences for a set of aptamers and one or more experimental measurements (e.g., fluorescence) for a characteristic of the aptamer as described in detail with respect to FIG. 2.

As described above, in some embodiments, the fine-tuned reward model(s) 335 (reward model) is an ensemble of a fine-tuned BERT model and a XGBoost which are both trained via supervised learning to predict a function-approximation metric for the characteristic of an aptamer. The results in Table 1 suggest that using the ensemble of these two models out-performs either one of the models on their own. However, as should be further understood from this data, regressing the fluorescence appears to generally achieve better performance than classifying after thresholding. Further, adding mono/di/tri-mer counts to features helps performance of the XGBoost model. Moreover, in general, the fine-tuned BERT regression model performed approximately as well as XGBoost regression model. Consequently, it should be further understood that it is contemplated that other types and variants of machine learning model may be implemented in other embodiments according to this disclosure such as an XGBoost classifier based model, XGBoost regression based model, a BERT classifier based model, and the like.

TABLE 1
II. Reward Model Training.

| Model | | | |
|---|---|---|---|
| Name | Details | Metrics | T-Stratified Metrics |
| XGB cf ≥ 6 | Features: | ≥6 ROC AUC: 0.856 | ≥6 ROC AUC: 0.559 |
| classifier | one-hot | ≥9 ROC AUC: 0.793 | ≥9 ROC AUC: 0.316 |
| | | Spearmanr: 0.690 | Spearmanr: 0.163 |
| XGB cf ≥ 9 | Features: | ≥6 ROC AUC: 0.856 | ≥6 ROC AUC: 0.599 |
| classifier | one-hot | ≥9 ROC AUC: 0.827 | ≥9 ROC AUC: 0.365 |
| | | Spearmanr: 0.655 | Spearmanr: 0.206 |
| XGB cf ≥ 9 | Features: | ≥6 ROC AUC: 0.902 | ≥6 ROC AUC: 0.576 |
| classifier | one-hot, | ≥9 ROC AUC: 0.917 | ≥9 ROC AUC: 0.426 |
| | mono/di/trimer | Spearmanr: 0.756 | Spearmanr: 0.290 |
| | counts | | |
| XGB | Features: | ≥6 ROC AUC: 0.913 | ≥6 ROC AUC: 0.592 |
| regression | one-hot, | ≥9 ROC AUC: 0.898 | ≥9 ROC AUC: 0.470 |
| | mono/di/trimer | Spearmanr: 0.768 | Spearmanr: 0.324 |
| | counts | | |
| XBG | Smaller | ≥6 ROC AUC: 0.902 | ≥6 ROC AUC: 0.600 |
| regression | model, n_ | ≥9 ROC AUC: 0.923 | ≥9 ROC AUC: 0.524 |
| | estimators = 20 | Spearmanr: 0.770 | Spearmanr: 0.350 |
| | gamma = 2, | | |
| | depth: 1 | | |
| BERT | Pre-trained + | ≥6 ROC AUC: 0.907 | ≥6 ROC AUC: 0.613 |
| regression | fine-tuned | ≥9 ROC AUC: 0.897 | ≥9 ROC AUC: 0.454 |
| | | Spearmanr: 0.797 | Spearmanr: 0.334 |
| Ensemble: | Ensemble by | ≥6 ROC AUC: 0.911 | ≥6 ROC AUC: 0.631 |
| XGB | averaging | ≥9 ROC AUC: 0.918 | ≥9 ROC AUC: 0.523 |
| regression + | outputs | Spearmanr: 0.803 | Spearmanr: 0.415 |
| BERT | | | |
| regression | | | |

The metrics shown in Table 1 were computed when training the reward model for designing 30 nucleotide TNA aptamers with Indol modified Thymine bases to target the Receptor Binding Domain (RBD) of the S1 Covid spike protein. The metrics were computed on a hold-out set of measurements made from a particle display assay and trained on the remaining data enforcing no sequences within 10 edits of each other between the train/test sets. The resulting test set was about 220 sequences. %

FIGS. 5A and 5B show scatter plots (top) and predicted receiver operating characteristic curves (ROC curves) (bottom) for a training set (left) and a test set (right) of actual fluorescence value (x-axis) and model predicted fluorescence (y-axis) for the BERT model (FIG. 5A) and XGBoost regression model (FIG. 5B). A ROC curve is a graphical plot that illustrates the prediction capability of a model as its discrimination threshold is varied. The ROC curve is created by plotting the true positive rate (TPR) against the false positive rate (FPR) at various threshold settings. The true-positive rate is also known as sensitivity, recall or probability of detection. The false-positive rate is also known as probability of false alarm and can be calculated as (1−specificity). The ROC curve is thus the sensitivity or recall as a function of fall-out (i.e., robustness). %

### Decoder Model Training

FIG. 6 shows a block diagram of a workflow 600 that uses predictions made by fine-tuned reward model(s) 335 described with respect to workflow 300 to train an unsupervised machine learning model such as a generative language model. The machine learning model is trained to take as input data: decoder parameters, an average reward target threshold, a sequence batch size, and aptamer sequence examples, and predict a batch of novel high-quality candidate aptamer sequences for aptamers that satisfy the average reward target threshold. The training approach is to pre-train the machine learning model using a large corpus of short non-coding RNA sequences so that the machine learning model learns transferable knowledge from these biologically/molecular related sequences in a pre-training phase 605. Thereafter, the machine learning model is fine-tuned algorithmically in a looped fine-tuning phase 610 comprising the following three steps: a) the machine learning model is used to generate a large batch (e.g., 128,000) of sequences based on input data, b) the reward model as described in detail with respect to FIG. 3 predicts a function-approximation metric for the characteristic of an aptamer associated with each of the generate sequences, which essentially scores each of the generated sequences, and c) the parameters of the machine learning model are updated via a single optimizer step so as to minimize a loss function based on the function-approximation metric or score associated with each of the generated sequences. Conceptually, the machine learning model is being fine-tuned on an infinitely large synthetic labeled dataset which is constructed dynamically by generating and scoring sequences.

In some embodiments, the machine learning model is a generative language model that utilizes a decoder-only transformer to predict a novel aptamer sequence where each nucleotide is predicted one at a time form left to right, conditioned autoregressively on the preceding nucleotides (i.e., predicts the next base given all of the preceding bases), based on patterns and structures within existing aptamer sequence data and the transferred information learned from a large corpus of ncRNA sequences in the pre-training phase 605. The generative language model uses neural networks to identify the patterns and structures within the existing aptamer sequence data to generate or design new and original aptamer sequences that satisfy the average reward target threshold.

As discussed above, one of the greatest challenges with training machine learning models to predict XNA aptamer characteristics (e.g., binding affinity) and design candidate sequences with optimized characteristics is the limited availability of data. Because of this, a transfer learning approach is utilized where prior to fine-tuning, generative language algorithms 615 (e.g., a decoder algorithm) are pre-trained in pre-training phase 605. The pre-training phase 605 comprises inputting a large corpus 625 into a pretrainer 620, the pretrainer 620 trains the generative language algorithms 615 using an unsupervised learning approach, and outputs pre-trained decoder model(s) 630. The pretrainer 620 is part of a machine learning operationalization framework (e.g., the prediction model training subsystem 202 described with respect to FIG. 2) comprising hardware such as one or more processors (e.g., a CPU, GPU, TPU, FPGA, the like, or any combination thereof), memory, and storage that operates software or computer program instructions (e.g., TensorFlow, PyTorch, Keras, and the like) to execute arithmetic, logic, input and output commands for training the generative language algorithms 615. As described above with respect to FIG. 3, the large corpus 625 comprises examples of short ncRNAs. By pre-training the generative language algorithms 615 via unsupervised learning on large datasets of non-coding RNAs, the generative language algorithms 615 learn transferable knowledge from ncRNA sequences such as patterns in the distribution of naturally occurring short single-stranded nucleobase molecules which serve a biological like protein binding. For example, how a sequence must be constituted in order to fold into a reliable secondary structure, or what features of a sequence enable it to interact with proteins.

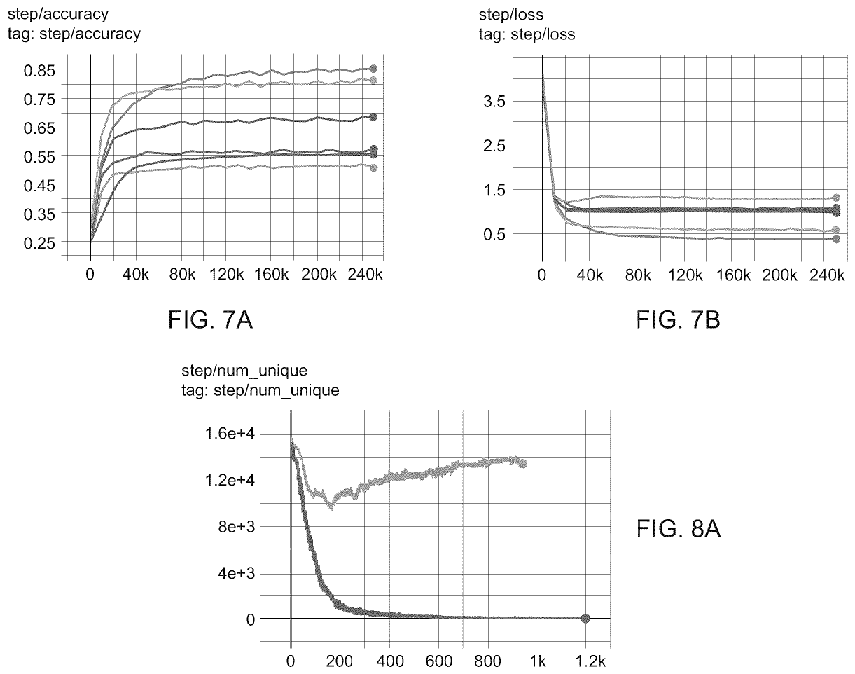

The pre-training of the generative language algorithms 615 is executed by the pretrainer 620 using a pre-training scheme such as MLM, CLM, or NSP. In some instances, the pre-training scheme is MLM. Under the MLM approach, a certain % of nucleobases in a given RNA sequence (e.g., 15%) from the large corpus 625 are randomly masked out and the generative language algorithms 615 are expected to predict those masked nucleobases based on context provided by surrounding nucleobases in that RNA sequence. Such a training scheme makes this model bidirectional in nature because the representation of the masked nucleobases is learnt based on the nucleobases that occur to its left as well as right. FIGS. 7A and 7B show pre-training results for exemplary 12 layer Decoder-only language model optimized for next base prediction on the non-coding RNA datasets. FIG. 7A shows masked base prediction accuracy and FIG. 7B shows loss over the course of 500,000 parameter update steps in pre-training. The curves show light blue: RNAcentral, orange: DASHR, red: miRBase mature, miRBase hairpin, pink: training, green: validation.

After having been pre-trained, the pre-trained decoder model(s) 630 are ready to be fine-tuned to generate (i.e., “design”) high quality candidate aptamer sequences to test via experimentation. This approach is also referred to as “transfer learning” where accuracy on a task (in this case design of an XNA aptamer sequence) is improved by first having learned a different but related task (e.g., masked nucleobase prediction (MLM) on datasets of non-coding RNAs). Fine-tuning the pre-trained decoder model(s) 630 to generate fine-tuned decoder model(s) 635 (Decoder Models) is a useful step in RLEF because it generates sequences which the reward model predicts to have a valuable characteristic (e.g., high affinity to the protein target). The fine-tuning procedure involves treating the pre-trained decoder model(s) 630 as though they were reinforcement learning agents, where the set of nucleotides generated so far is the “state”, the next nucleotide chosen to come next in the sequences is the “action” and the final sequences' predicted target binding affinity from the reward model is the “reward”.

The pre-trained decoder model(s) 630 are fine-tuned algorithmically (see Algorithm 1 RLHF in FIG. 6) in the looped fine-tuning phase 610. The fine-tuning phase 610 comprises the decoder fine tuner 640 inputting data 645 into the pre-trained decoder model(s) 630, the decoder fine tuner 640 fine-tuning the pre-trained decoder model(s) 630 using an unsupervised learning approach to generate high quality candidate aptamer sequences 650, and the decoder fine tuner 640 outputs fine-tuned decoder model(s) 635. The decoder fine tuner 640 is also part of the machine learning operationalization framework (e.g., the prediction model training subsystem 202 described with respect to FIG. 2) comprising hardware such as one or more processors (e.g., a CPU, GPU, TPU, FPGA, the like, or any combination thereof), memory, and storage that operates software or computer program instructions (e.g., TensorFlow, PyTorch, Keras, and the like) to execute arithmetic, logic, input and output commands for fine-tuning the pre-trained decoder model(s) 630.

The input data 645 comprises decoder parameters, an average reward target threshold, a sequence batch size, and examples of known aptamer sequences that have one or more characteristics (e.g., a binding affinity for a given target). The decoder parameters are model parameters that determine how the pre-trained decoder model(s) 630 processes the known aptamer sequences and how it generates predictions for novel aptamer sequences. The decoder parameters play an important role in controlling the output of the pre-trained decoder model(s) 630. For example, the parameters can control generation of the aptamer sequence such that the associated aptamer has various characteristics such as binding affinity, dissociation kinetics, nuclease stability, and the like. The average reward target threshold is a threshold on a value of the proxy for the characteristic that defines whether the aptamer sequences output on average by the fine-tuned decoder model(s) 635 would be considered high-quality candidate aptamer sequences (i.e., aptamer sequences with a fluorescence predicted to be greater than the average reward target threshold would be considered high-quality candidate aptamer sequences). The sequence batch size is a size or number of sequences to be generated by the pre-trained decoder model(s) 630 per batch of examples (e.g., 10,000 sequences). In some instances, the pre-trained decoder model(s) 630 output a large batch (e.g., 100,000-2000,000) of aptamer sequences 650.

The input data 645 is fed into the pre-trained decoder model(s) 630. The pre-trained decoder model(s) 630 generates aptamer sequences 650 that satisfy one or more characteristics such as high-predicted-affinity and/or high-predicted-specificity (e.g., can bind a given target with a certain level of binding affinity) based on the decoder parameters, the average reward target threshold, the sequence batch size, and examples of known aptamer sequences. The pre-trained decoder model(s) 630 generates each of the aptamer sequences 650 by choosing each nucleotide one at a time from left-to-right, conditioning auto-regressively on the preceding nucleotides. The pre-trained decoder model(s) 630 may identify hundreds to thousands of additional or alternative sequences, for example, a large batch (e.g. 128,000) of aptamer sequences.

The aptamer sequences 650 generated by the pre-trained decoder model(s) 630 are then input into a fine-tuned reward model 335, as described with respect to FIG. 3. The fine-tuned reward model 335 predicts a function-approximation metric for one or more characteristics of each of the aptamer sequences 650. The function-approximation metric can be stored in a data table 655 associated with each of the aptamer sequences 650 for downstream processing such as analysis of model performance and use as a reward for fine-tuning the pre-trained decoder model(s) 630. For example, an average reward can be calculated by taking the average of the function-approximation metric predicted for a characteristic of each of the aptamer sequences 650. When the average reward is less than the average reward target threshold, the decoder parameters of the pre-trained decoder model(s) 630 are updated using the rewards in a reinforcement learning approach. When the average reward is equal to or greater than the average reward target threshold, the pre-trained decoder model(s) 630 are considered fine-tuned and output as the fine-tuned decoder model(s) 635 for deployment or use in a production environment (inference phase). Conceptually, the pre-trained decoder model(s) 630 are being trained on an infinitely large synthetic labeled dataset which is constructed dynamically by generating aptamer sequences 650 and scoring sequences with a function-approximation metric.

The reinforcement learning approach utilizes a loss function 660 and single optimizer step such as Stochastic Gradient Descent or ADAM so as to minimize the loss function 660. In some instances, the reinforcement learning approach includes iterative operations (i.e., generating sequences, generating scores for the sequences, and using the scores as rewards for optimizing a set of parameters) to find a set of parameters for the pre-trained decoder model(s) 630 that minimizes the loss function 660. Each iteration can involve finding a set of parameters for the pre-trained decoder model(s) 630 so that the value of the loss function 660 using the set of parameters is smaller than the value of the loss function 660 using another set of parameters in a previous iteration. The loss function 660 can be constructed to optimize the pre-trained decoder model(s) 630 to generate higher scoring sequences. Once the set of parameters are identified that allow for the pre-trained decoder model(s) 630 to generate aptamer sequences 650 having an average score equal to or greater than the average reward target threshold, the pre-trained decoder model(s) 630 has been trained and can be tested, validated, and/or utilized for generation as designed. The loss function 660 comprises three components: a proximal policy optimization 665 (first loss), a sequence entropy bonus 670 (second loss), and a Kullback-Leibner (KL) divergence objective function 675 (third loss).

The proximal policy optimization 665 component is responsible for optimizing the pre-trained decoder model(s) 630 to generate higher scoring sequences and is defined by an objective such as the clipped surrogate objective specified by Equation 7 of Proximal Policy Optimization (PPO) (see Schulman, John et al. “Proximal Policy Optimization Algorithms.” ArXiv abs/1707.06347 (2017): n. pag.) This first loss component is designed to optimize sequential decision making to maximize the reward, (i.e., the score from the reward model). The sequential decision process refers to the process of deciding which base will come next from left to right in the aptamer sequence. The objective comprises sampling the actions (i.e., a nucleotide chosen to come next in the aptamer sequence). The actions have corresponding sampling probabilities and differences in the reward predicted for each of the novel aptamer sequences are measurable and associable to the actions. Ultimately, generating higher scoring aptamer sequences.

Nonetheless, it is common for the pre-trained decoder model(s) 630 to “over optimize” the fine-tuned reward model 335 and end up just outputting a single high scoring sequence. This is undesirable because there is often a need to test an entire diverse pool of high-scoring sequences. The sequence entropy bonus 670 component rewards the model for, loosely speaking, being ambivalent about what base could come next, and penalizes the model for being too confident about the entire sequence. The sequence entropy bonus 670 component is computed based on the sampling probabilities and differences in the reward predicted for each of the aptamer sequences. Mors specifically, the sequence entropy bonus 670 component is configured to compute entropy of a next nucleotide or action prediction distributions output by the pre-trained decoder model(s) 630, summed over an entire aptamer sequence, as shown in Equation (1). The model parameters of the pre-trained decoder model(s) 630 are adjusted based on the proximal policy optimization 665 and the sequence entropy bonus 670 to finetune the pre-trained decoder model(s) 630 for generating subsequent aptamer sequences having a more desirable measurement for the characteristic.

$$\begin{matrix}III . & \\ {\sum }_{i = 1}^{N} - log {\sum }_{j = 1}^{L} Entr ( {x}_{i , j} ) & Equation ( 1 )\end{matrix}$$

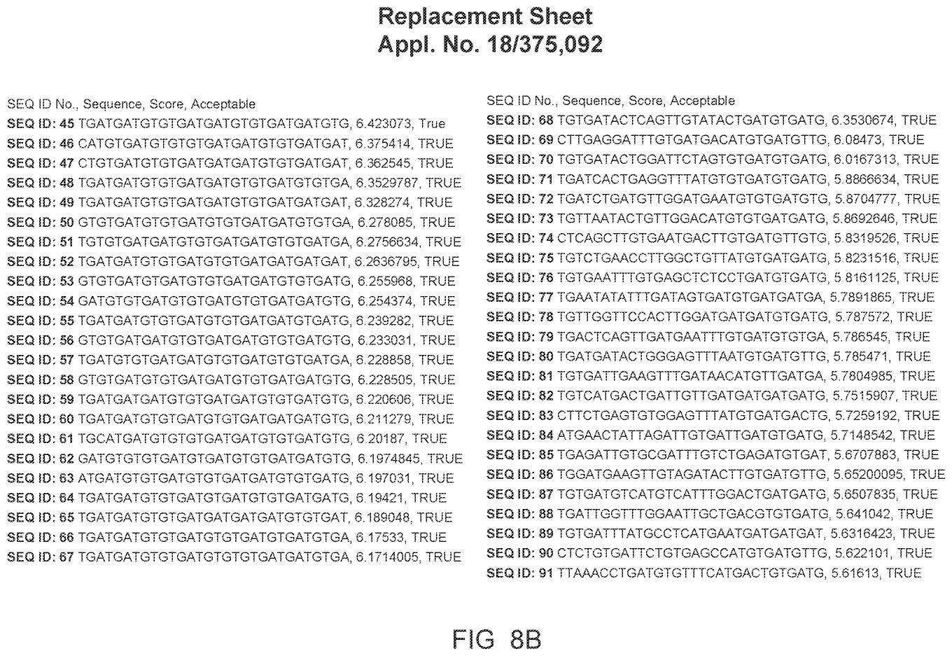

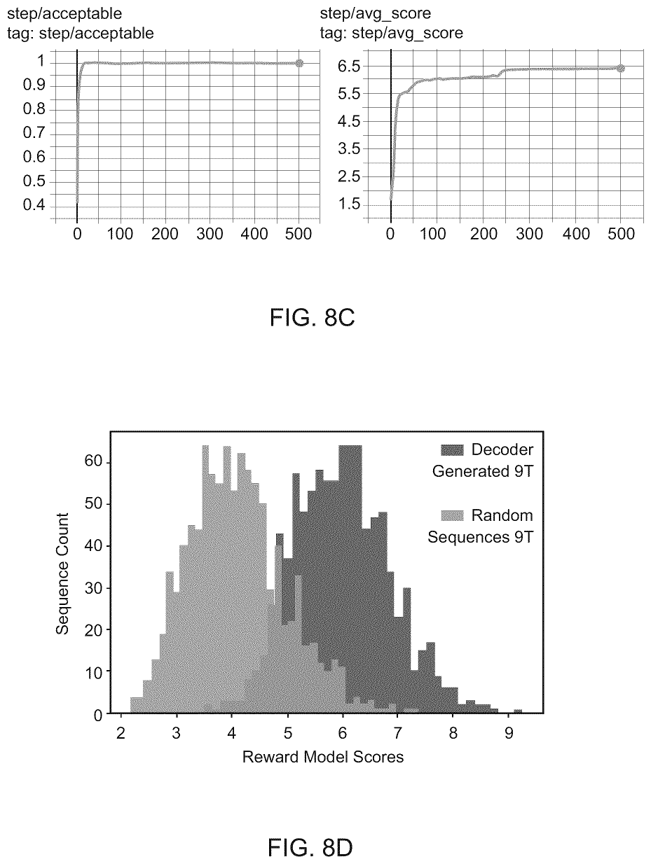

IV Where x_{i,j} is the next-token prediction distribution logits produced by the pre-trained decoder model(s) 630 at nucleotide position j in sequence i. Then the −log non-linearity is applied after summing entropy over the entire sequence to capture higher sequence level entropy, rather than globally higher single-base entropy. This loss is effective at generating a more diverse pool of candidate aptamers as shown in FIG. 8A. FIG. 8A shows the total number of unique sequences generated by the pre-trained decoder model 630 from a pool of 64,000 generated sequences with (orange) and without (blue) the Sequence Entropy Bonus 460 loss. Without this loss component, the pre-trained decoder model 630 degenerates into outputting the same sequence repeatedly.

As another measure to prevent over optimization/exploitation of the fine-tuned reward model 335, a penalty is included for divergence of the next-base prediction from the pre-trained model's distribution using Kullback-Leibner (KL) divergence objective function 675. Recall, as described herein, the pre-trained decoder model 630 captures the distribution of protein interacting non-coding RNA sequences. So, in essence the KL divergence objective function 675 is configured to encourage generation of “biologically plausible” sequences. To do this, the KL divergence objective function 675 computes loss based on the sampling probabilities and differences in the reward predicted for each of the aptamer sequences by applying a penalty for divergence of a next nucleotide or action prediction from a distribution of the pre-trained decoder model 630. FIG. 8B shows this is effective at avoiding degenerate looking sequences. More specifically, FIG. 8B shows top-scoring generated sequences from the pre-trained decoder model 630 fine-tuned with RLEF before (left) and after (right) the addition of the KL divergence objective function 675, which penalizes divergence from the pre-trained distribution of non-coding RNAs as learned by the pre-trained decoder model 630. Although scores are generally lower with the KL divergence objective function 675, the sequences are much more diverse and avoid degenerating into repeating patterns of bases like repeated “GT” as seen on the left.

In addition to maximizing the reward, the reinforcement learning approach can be further configured with a constraint scheme to enforce that the pre-trained decoder model 630 learns to generate XNA sequences that can be practically synthesized. This constraint scheme can be enforced by removing the reward when the pre-trained decoder model 630 generates a nucleotide that renders the sequence unsynthesizable. Unsynthesizable sequences may be defined to include those that comprise more than three repeat bases in a row, a GC content higher than 50%, too many functional bases (thymine modified by indol or benzyl chemical groups)—thus placing a limit on the amount of functionalized bases, or any combination thereof. Consequently, those sequences that do not include one or more of these criteria/constraints are identifiable as synthesizable sequences. FIG. 8C shows that with this constraint scheme the pre-trained decoder model 630 quickly learns to generate synthesizable sequences which are also predicted to be high for the given characteristic-binding affinity. More specifically, FIG. 8C shows RLEF training run metrics over the course of 500 generation/update steps for fine-tuning a 12 layer Decoder generative model for S1 RBD/Benzyl TNA sequences. Left shows the proportion of generated sequences which pass the synthesizability criteria at each step. It can be observed that the pre-trained decoder model 630 quickly learns to generate only synthesizable sequences in ˜20 steps. Right shows average rewards model scores over the course of RLEF fine-tuning. It can be further observed that the pre-trained decoder model 630 learns to generate sequences which the fine-tuned reward model 335 scores on average >6 fluorescence, which if it occurred in the actual assay, would be considered a “hit”. FIG. 8D shows a graph of the scores given by the fine-tuned reward model 335 for random sequences (orange; left) in comparison with sequences generated from the fine-tuned decoder model(s) 635 (blue; right).

### EXAMPLES

The following examples are offered by way of illustration, and not by way of limitation.

### 1. Implementation/Execution

Initially, a Colab Notebook implementation was used to generate sequences for plate 17. Colab notebooks are Jupyter notebooks that run in the cloud and are highly integrated with Google Drive, making them easy to set up, access, and share. The RLEF based sequence design method described herein was prototyped in the Colab Notebook implementation to test in Hydrogel Particle Display Plates targeting S1 RBD with Indol in plate 17 in the batch of plates 16, 17, 18. Thereafter, the Colab Notebook implementation was translated into a distributed environment version that runs on TPU pods, starting with an implementation in experimental and later an official implementation in the main code base. This enabled scaling up the reward and decoder models to be very large models and using very large batch sizes.

### 2. RLEF Experimental Results

Colab Notebook implementation with Results Analysis—The efficacy of the RLEF based sequence design method was tested with S1 RBD in hydrogel PD plate 17. The plate was designed with two tranches:
40 sequences: Reward Model Only+Random Search
40 sequences: RLEF-Reward Model+Decoder Model

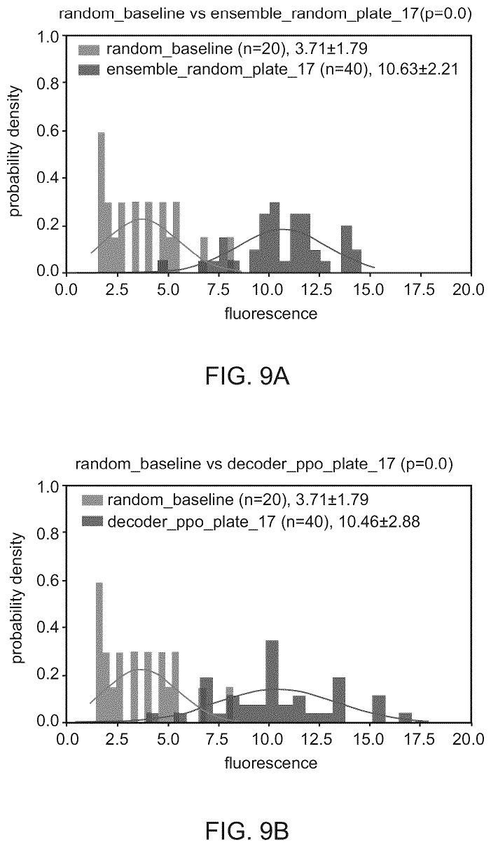

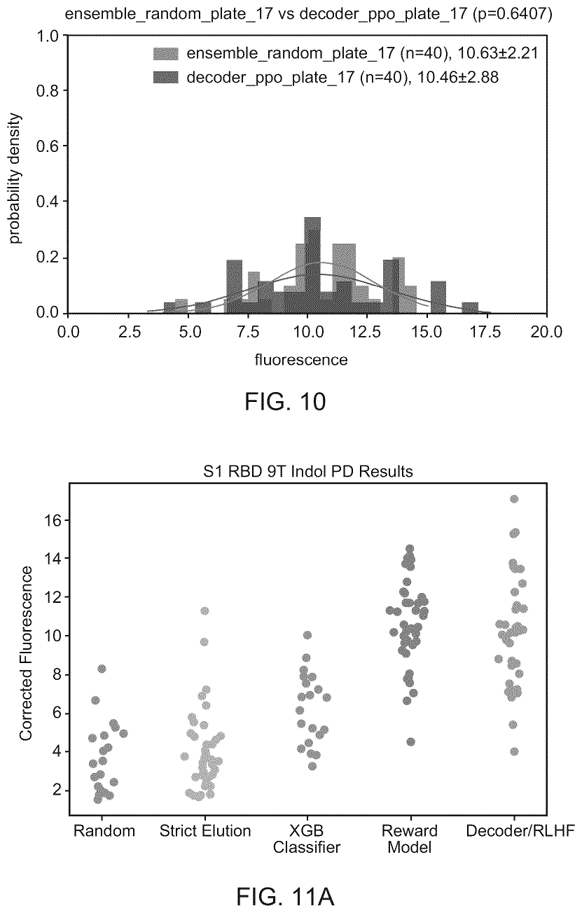

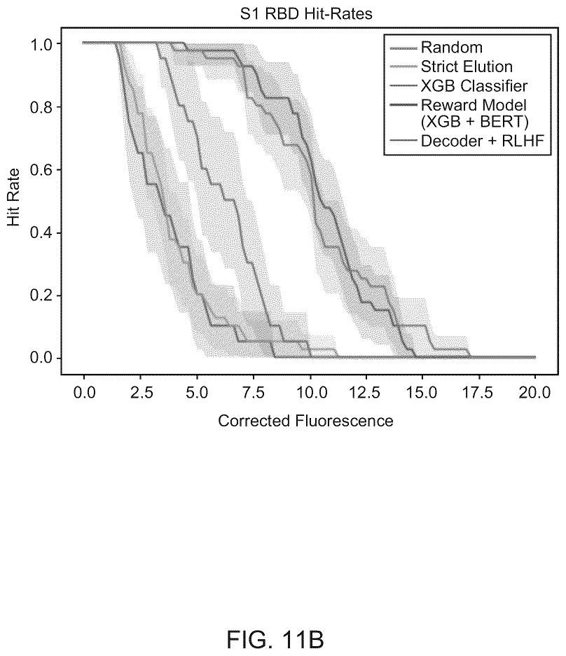

V. The results suggest that choosing high-scoring sequences as per the reward model ensemble is effective in designing aptamers with one or more desired characteristics such as high affinity aptamers (FIG. 9A). In addition, RLEF was demonstrated as also being effective in designing aptamers with one or more desired characteristics such as high affinity aptamers (FIG. 9B). FIGS. 9A and 9B show results of statistical tests comparing RLEF tuned decoder (blue, right) and decoder-ablation (blue, left) against random baseline. As shown, the Reward Model Only+Random Search and RLEF generated sequences both significantly out-perform the random baseline. FIG. 10 shows a comparison between RLEF generated sequences versus randomly searching for high-scoring sequences. FIGS. 11A and 11B show in comparison with other methods/baselines, the RLEF approach finds high affinity (e.g. ≥9 corrected fluorescence) on the 9T modality with significantly higher success rate as compared with the previous art of XGBoost classifier+random search (FIG. 11B). As shown, at the very upper end, the highest fluorescence/affinity sequences are generated via the RLEF method (highest purple dots in FIG. 11A).

The following sequences in Table 2 are examples which were designed with the RLEF method and tested experimentally (positive results described above).

TABLE 2
VI.

| | | Aptamer | | | | |
|---|---|---|---|---|---|---|
| | Aptamer to | Sequence to | Sequence | | Validation | Validation |
| Well | Validate | Order | Intent | Description | Language | Experiment |
| A1 | TAAAAGAGAT | TTTAATTATA | NEGATIVE | Negative | TNA | HYDROGEL |
| | CGTGCGCCCA | TGGGCGCACG | CONTROL | control | | PRIMER |
| | TATAATTAAA | ATCTCTTTTA | | | | EXTENSION |
| | (SEQ ID | (SEQ ID | | | | |
| | NO: 1) | NO: 23) | | | | |
| A2 | TATGTTGTTC | TGACTTTGGG | POSITIVE | Control | TNA | HYDROGEL |
| | GTCGAGCATA | TATGCTCGAC | CONTROL | ERG794 | | PRIMER |
| | CCCAAAGTCA | GAACAACATA | | | | EXTENSION |
| | (SEQ ID | (SEQ ID | | | | |
| | NO: 2) | NO: 24) | | | | |
| A3 | ACAAACTTCC | CGTGTACAAC | BINDER | Ensemble | TNA | HYDROGEL |
| | CGCGTTGTCT | AGACAACGCG | | random | | PRIMER |
| | GTTGTACACG | GGAAGTTTGT | | | | EXTENSION |
| | (SEQ ID | (SEQ ID | | | | |
| | NO: 3) | NO: 25) | | | | |
| A4 | ATGGCCCGAC | TGTTGGGAGA | BINDER | Decoder ppo | TNA | HYDROGEL |
| | ATGTTGTTGT | ACAACAACAT | | | | PRIMER |
| | TCTCCCAACA | GTCGGGCCAT | | | | EXTENSION |
| | (SEQ ID | (SEQ ID | | | | |
| | NO: 4) | NO: 26) | | | | |
| A5 | GAGAGTTGTT | CCCTTTGACA | BINDER | Ensemble | TNA | HYDROGEL |
| | TGTTCCAAGG | CCTTGGAACA | | random | | PRIMER |
| | TGTCAAAGGG | AACAACTCTC | | | | EXTENSION |
| | (SEQ ID | (SEQ ID | | | | |
| | NO: 5) | NO: 27) | | | | |
| A6 | CCTCGCTGTA | TTTGGGATGG | BINDER | Decoder ppo | TNA | HYDROGEL |
| | ACTGTTGTCT | AGACAACAGT | | | | PRIMER |
| | CCATCCCAAA | TACAGCGAGG | | | | EXTENSION |
| | (SEQ ID | (SEQ ID | | | | |
| | NO: 6) | NO: 28) | | | | |
| A7 | ACACATTCCA | CGTTGACAGT | BINDER | Ensemble | TNA | HYDROGEL |
| | TGTTGTCCCT | AGGGACAACA | | random | | PRIMER |
| | ACTGTCAACG | TGGAATGTGT | | | | EXTENSION |
| | (SEQ ID | (SEQ ID | | | | |
| | NO: 7) | NO: 29) | | | | |
| A8 | CGATCGTGTT | TTGGTGGGAA | BINDER | Decoder ppo | TNA | HYDROGEL |
| | CAACTGTGTG | CACACAGTTG | | | | PRIMER |
| | TTCCCACCAA | AACACGATCG | | | | EXTENSION |
| | (SEQ ID | (SEQ ID | | | | |
| | NO: 8) | NO: 30) | | | | |
| A9 | AGTGTTGTTG | CCTTCGCTGT | BINDER | Ensemble | TNA | HYDROGEL |
| | TTATAAGTAG | CTACTTATAA | | random | | PRIMER |
| | ACAGCGAAGG | CAACAACACT | | | | EXTENSION |
| | (SEQ ID | (SEQ ID | | | | |
| | NO: 9) | NO: 31) | | | | |
| A10 | AACAGGCTTC | TTGGCGGGAC | BINDER | Decoder ppo | TNA | HYDROGEL |
| | ACTGTTCTTT | AAAGAACAGT | | | | PRIMER |
| | GTCCCGCCAA | GAAGCCTGTT | | | | EXTENSION |
| | (SEQ ID | (SEQ ID | | | | |
| | NO: 10) | NO: 32) | | | | |
| A11 | GACCATTGTT | GGTGTTGCGT | BINDER | Ensemble | TNA | HYDROGEL |
| | ATGTTGTATC | GATACAACAT | | random | | PRIMER |
| | ACGCAACACC | AACAATGGTC | | | | EXTENSION |
| | (SEQ ID | (SEQ ID | | | | |
| | NO: 11) | NO: 33) | | | | |
| A12 | ACGCGTGTTC | TTTGTTGGAG | BINDER | Decoder ppo | TNA | HYDROGEL |
| | TGCTGTCTTC | GAAGACAGCA | | | | PRIMER |
| | CTCCAACAAA | GAACACGCGT | | | | EXTENSION |
| | (SEQ ID | (SEQ ID | | | | |
| | NO: 12) | NO: 34) | | | | |
| B1 | ACAACAGCGT | CGACGGGTTA | BINDER | Ensemble | TNA | HYDROGEL |
| | GTTGTTGTGT | ACACAACAAC | | random | | PRIMER |
| | TAACCCGTCG | ACGCTGTTGT | | | | EXTENSION |
| | (SEQ ID | (SEQ ID | | | | |
| | NO: 13) | NO: 35) | | | | |
| B2 | ATCAACATGT | GGCTGGATGG | BINDER | Decoder ppo | TNA | HYDROGEL |
| | ATGTGTGTTC | GAACACACAT | | | | PRIMER |
| | CCATCCAGCC | ACATGTTGAT | | | | EXTENSION |
| | (SEQ ID | (SEQ ID | | | | |
| | NO: 14) | NO: 36) | | | | |
| B3 | ACCCGCCTTG | GTGCTTACAA | BINDER | Ensemble | TNA | HYDROGEL |
| | TAACTTGTCA | TGACAAGTTA | | random | | PRIMER |
| | TTGTAAGCAC | CAAGGCGGGT | | | | EXTENSION |
| | (SEQ ID | (SEQ ID | | | | |
| | NO: 15) | NO: 37) | | | | |
| B4 | CACCTTCGCA | TGTGGGACGA | BINDER | Decoder ppo | TNA | HYDROGEL |
| | ATCACTTGTT | AACAAGTGAT | | | | PRIMER |
| | TCGTCCCACA | TGCGAAGGTG | | | | EXTENSION |
| | (SEQ ID | (SEQ ID | | | | |
| | NO: 16) | NO: 38) | | | | |
| B5 | CGCAATGTTG | CGTGAAGGTG | BINDER | Ensemble | TNA | HYDROGEL |
| | AACCGTTGTT | AACAACGGTT | | random | | PRIMER |
| | CACCTTCACG | CAACATTGCG | | | | EXTENSION |
| | (SEQ ID | (SEQ ID | | | | |
| | NO: 17) | NO: 39) | | | | |
| B6 | GCCCGTTTAA | TGTGCGGGAA | BINDER | decoder ppo | TNA | HYDROGEL |
| | CATGTTCTAC | GTAGAACATG | | | | PRIMER |
| | TTCCCGCACA | TTAAACGGGC | | | | EXTENSION |
| | (SEQ ID | (SEQ ID | | | | |
| | NO: 18) | NO: 40) | | | | |
| B7 | AGCCACCACT | CTCGAACAGC | BINDER | Ensemble | TNA | HYDROGEL |
| | GTTGTTGTAA | TTACAACAAC | | random | | PRIMER |
| | GCTGTTCGAG | AGTGGTGGCT | | | | EXTENSION |
| | (SEQ ID | (SEQ ID | | | | |
| | NO: 19) | NO: 41) | | | | |
| B8 | CGACCTGTTC | TTGGGTGGGA | BINDER | Decoder ppo | TNA | HYDROGEL |
| | ATTCCTATGT | ACATAGGAAT | | | | PRIMER |
| | TCCCACCCAA | GAACAGGTCG | | | | EXTENSION |
| | (SEQ ID | (SEQ ID | | | | |
| | NO: 20) | NO: 42) | | | | |
| B9 | AACCCAATTG | TGTCGGCAAA | BINDER | Ensemble | TNA | HYDROGEL |
| | TCATGTGTCC | GGACACATGA | | random | | PRIMER |
| | TTTGCCGACA | CAATTGGGTT | | | | EXTENSION |
| | (SEQ ID | (SEQ ID | | | | |
| | NO: 21) | NO: 43) | | | | |
| B10 | CGCCTAAGTA | TTCGGGCGAA. | BINDER | Decoder ppo | TNA | HYDROGEL |
| | TGTCACTTGT | ACAAGTGACA | | | | PRIMER |
| | TTCGCCCGAA | TACTTAGGCG | | | | EXTENSION |
| | (SEQ ID | (SEQ ID | | | | |
| | NO: 22) | NO: 44) | | | | |
| B11 | TAAAAGAGAT | TTTAATTATA | NEGATIVE | Negative | TNA | HYDROGEL |
| | CGTGCGCCCA | TGGGCGCACG | CONTROL | control | | PRIMER |
| | TATAATTAAA | ATCTCTTTTA | | | | EXTENSION |
| | (SEQ ID | (SEQ ID | | | | |
| | NO: 1) | NO: 23) | | | | |
| B12 | TATGTTGTTC | TGACTTTGGG | POSITIVE | Control | TNA | HYDROGEL |
| | GTCGAGCATA | TATGCTCGAC | CONTROL | ERG794 | | PRIMER |
| | CCCAAAGTCA | GAACAACATA | | | | EXTENSION |
| | (SEQ ID | (SEQ ID | | | | |
| | NO: 2) | NO: 24) | | | | |

### End-to-End Pipeline to Identify and Generate Response to a Query

FIG. 12 shows a block diagram of a pipeline 1200 for strategically identifying and generating aptamers with a one or more characteristics (e.g., high affinity binders of molecular targets). In various embodiments, the pipeline 1200 implements in vitro experiments and in silico computation and machine learning based techniques to iteratively improve a process for identifying aptamers with a one or more characteristics that can bind any given molecular target. At block 1205, in vitro binding selections (e.g., phages display or SELEX) are performed where a given molecular target (e.g., a protein of interest) is exposed to tens of trillions of different potential binders (e.g., a library of 10¹⁴-10¹⁵ nucleic acid aptamers), a separation protocol is used to remove non-binding aptamers (e.g., flow-through), and the binding aptamers are eluted from the given target. The binding aptamers and/or the non-binding aptamers are sequenced to identify what aptamers do and/or do not bind the given target. This binding selection process may be repeated for any number of cycles (e.g., 1 to 3 cycles) to reduce the absolute count of potential aptamers from tens of trillions of different potential aptamers down to millions or trillions of sequences 1210 of aptamers identified to have some level of binding (specific and non-specific) for the given target.

At block 1215, the sequences of binding aptamers, non-binding aptamers, or a combination thereof obtained from block 1205 are used to train a machine learning algorithm (e.g., a highly parameterized machine algorithm with a parameter count of greater than or equal to 10,000, 30,000, 50,000, or 75,000) and learn a fitness function capable of ranking the fitness (quality) of sequences of aptamers based on one or more characteristics such as a design criteria proposed for an aptamer, a problem being solved (e.g., finding an aptamer that is capable of binding to target with a high-affinity), and/or an answer to a query (e.g., what aptamers are capable of inhibiting function A). In some instances, the sequences of binding aptamers, non-binding aptamers, or a combination thereof are labeled with one or more sequence properties.

The one or more sequence properties may include a binding-approximation metric that indicates whether an aptamer included in or associated with the training data bound to a particular target. The binding-approximation metric can include (for example) a binary value or a categorical value. The binding-approximation metric can indicate whether the aptamer bound to the particular target in an environment where the aptamer and other aptamers (e.g., other potential aptamers) are concurrently introduced to the particular target. The binding-approximation metric can be determined using a high-throughput assay, such as in vitro binding selections (e.g., phages display or SELEX), a low-throughput assay, such as in vitro Bio-Layer Interferometry (BLI), or a combination thereof.

Additionally or alternatively, the one or more sequence properties may include a functional-approximation metric that indicates whether an aptamer included in or associated with the training data functions as intended (e.g., inhibits function A). The functional-approximation metric can include (for example) a binary value or a categorical value. The function-approximation metric can be determined using a low-throughput assay, such as an optical fluorescence assay or any other assay capable of detecting functional changes in a biological system as described with respect to FIG. 2 (e.g., inhibiting an enzyme, inhibiting protein production, promoting binding between molecules, promoting transcription, etc.). Further, the function-approximation metric may be used to infer the binding-approximation metric (e.g., if function A is inhibited it can be inferred that the molecule bound to the particular target).

Machine learning algorithms are procedures that are implemented in computer code and uses data (e.g., experimental results) to generate machine learning models. The machine learning models represent what was learned by the machine learning algorithms during training. In other words, the machine learning models are the data structures that are saved after running machine learning algorithms on training data and represents the rules, variables, and any other algorithm-specific data structures required to make predictions. The use of a large data set with diverse sequences of binding aptamers (e.g., millions or trillions of binders) in the training allows for the algorithm to learn all of the parameters required for estimating the fitness of aptamer candidates for a given problem. Otherwise, the problem of having a large number of parameters and dimensions yet small data sets results in overfitting, which means the learned function is too closely fit to a limited set of data points and works only for the data set the algorithm was trained with, rendering the learned parameters pointless.

The machine learning model in block 1215 trained on the large data set from block 1205 can then take new input sequences not necessarily discovered in an in vitro binding selection experiment and estimate a fitness for those input sequences given one or more characteristics (e.g., finding an aptamer that is capable of binding to a given target with a high affinity). The new input sequences may be generated using a machine learning algorithm (e.g., an algorithm in block 1215). In some instances, the machine learning model in block 1215 may include a genetic algorithm to generate new aptamers based on evolutionary models from one or more aptamers from binding selection experiments (e.g., from block 1205). Thus, model(s) in block 1215 may artificially increase the search space for aptamers that can bind the target and solve the given problem. The search space may be increased from the 10¹⁴-10¹⁵ nucleic acid aptamers investigated in the in vitro experimentation stage to at least 10²⁴ nucleic acid aptamers and beyond, depending on algorithm complexity and available computational resources.

The sequences of binding aptamers, non-binding aptamers, or a combination thereof obtained from block 1205 are going to have a low signal to noise ratio (and low label quality). In other words, the sequences in block 1210 may include a small number of sequences of aptamers with specific binding or high affinity (signal) and a large amount of aptamer sequences with non-specific binding or low affinity binding to the given target (noise). Essentially, the signal to noise ratio is a fraction of tested aptamers that have the desired binding characteristics when assayed with high/low throughput characterization or validation. Typically, machine learning algorithms may model both signal and noise, or a relationship thereof. In other words, the model may include the two parts of the training data—the underlying generalizable truth (the signal), and the randomness specific to that dataset (the noise). Fitting both of those parts can increase the training set accuracy, but fitting the signal also increases test set accuracy or generalization (and real-world performance) while fitting the noise decreases both the test set accuracy and real-world performance (causes overfitting). Thus, conventional regularization techniques such as L1 (lasso regression), L2 (ridge regression), dropout, and the like may be implemented in the training to make it harder for the algorithm to fit the noise, and so more likely for the algorithm to fit the signal and generalize more accurately.

However, conventional regularization techniques can lead to dimensionality reduction, which means the machine learning model is built using a lower dimensional dataset (e.g., less parameters). This can lead to a high bias error in the outputs (known as underfitting). In order to overcome these challenges and others, aspects of the present disclosure are directed to using a combination of in silico computational and machine learning based techniques (e.g., ensemble of neural nets, genetic search processes, regularized regression models, linear optimization, and the like) in combination with various in vitro experimentation techniques (e.g., binding selections, SELEX, and the like) to identify or design markedly different sequences with better properties, while maintaining sufficient predictive power to align on a small set of sequences (e.g., tens to hundreds) appropriate for low-throughput characterization or validation.

These various machine learning and experimentation techniques are implemented in the pipeline 1200 via a aptamer development architecture (e.g., the exemplary architecture shown in FIG. 12 and described herein) to decrease the absolute number of sequences being used as input for each stage while increasing the signal to noise ratio (e.g., decreasing the noise) and label quality (e.g., quality of the binding-approximation metric and/or functional-approximation metric, and to ultimately predict the highest quality binders (e.g., highest-affinity) for any given molecular target. In some instances, a machine learning algorithm used in block 1215 is a series of algorithms such as a neural network (i.e., a parameter count of greater than or equal to 10,000, 30,000, 50,000, or 75,000). A series of algorithms offers increased flexibility and can scale in proportion to the amount of training data available. A tradeoff for this flexibility may be that the algorithms learn via a stochastic training algorithm which means that the algorithms are sensitive to both the specific training data set (presumed to be a random sample from some fixed distribution) and also the initial conditions, etc., of the training run (e.g., seeds for pseudo-random number generators). Additionally, there is also randomness that is hard to control for even if random seeds are set because modern GPUs (presumably TPUs) are not guaranteed deterministic. This means that the algorithms are subject to overfitting and can have high variance when it comes to making a final prediction (e.g., prediction of a fitness score for additional or alternative sequences of aptamers).

In order to overcome this variance, in some instances, the machine learning algorithm is configured as a series of multiple neural networks trained using an ensemble-based approach to combine the predictions from the multiple neural networks. Combining the predictions from multiple neural networks counters the variance of a single trained neural network model and can reduce generalization error (also known as the out-of-sample error, which is a measure of how accurately an algorithm is able to predict outcome values for previously unseen data). For example, generalization error is typically decomposed into bias and variance; bias is (roughly) reduced by more expressive models (e.g., neural nets with many more parameters) but increasing the flexibility of models can lead to overfitting. Variance is (roughly) reduced by ensembles or larger datasets. Thus, for instance, random forests are ensembles of very flexible models (decisions trees)—the low bias of the component models usually lead to high variance solutions, so this can be counteracted by using an ensemble of trees, each fit to a random subset (optionally along with other techniques) of the data. The results of the ensemble of neural networks are predictions that are less sensitive to the specifics of the training data, choice of training scheme, and the randomness inherent in a single training run.

The trained machine learning model (e.g., an ensemble of neural networks) may then be used to perform a search process (e.g., genetic search) to identify sequences that have a high predicted fitness score. The sequences of aptamers identified by the model(s) in block 1215 may then be output, as shown in block 1220. The output from block 1220 may comprise thousands to millions of aptamer sequences. These sequences may be novel sequences that could not be discovered using a binding selection experiment.

In some instances, the search process in block 1215 is a genetic search process that uses a genetic algorithm, which mimics the process of natural selection, where the fittest individuals (e.g., aptamers with a potential for binding a given target) are selected for reproduction in order to produce offspring of the next generation (e.g., aptamers with the greatest potential for binding the given target). If parents have better fitness, their offspring will be better than parents and have a better chance at surviving. This process may continue iterating until a generation with the fittest individuals are found. Therefore, the aptamers in block 1220 (e.g., thousands of sequences) may have high probability or potential for satisfying the one or more characteristics (e.g., binding the given target with high affinity). In certain instances, the genetic algorithm is constrained to a limited number of nucleotide edits away from the training dataset knowing that variance of empirical labels relative to highly parameterized machine learning model predictions increases drastically. The model may stop the search process when another criterion such as maximum number of predicted aptamers is met.

At block 1225, identified or designed (e.g., by genetic search algorithm) sequences of aptamers from block 1220 may be used to synthesize aptamers, which are used for subsequent binding selections. The aptamer sequences generated by the described computational method are used to physically synthesize aptamers by solid phase oligonucleotide synthesis or similar wet-lab aptamer synthesis processes.

Solid phase oligonucleotide synthesis comprises a chemical process by which solid supports (e.g., resins, controlled pore glass, and polystyrene as examples) are used to anchor oligonucleotides during synthesis. In addition to the solid support, modified nucleosides known as phosphoramidtes serve as building blocks, or “bases” for oligonucleotide synthesis, where the modified nucleosides comprises one or more protection groups. Protection groups are defined as chemical groups that prevent unintended side reactions from occurring but are easily removed when necessary. Typically, phosphoramidite monomers comprise 4 specific protection groups: (i) a dimethoxytrityl (DMT) group which protects the 5′ hydroxyl group for the deoxyribose sugar, (ii) a diisopropylamino group that serves as the leaving group during azole catalyst when the next phosphoramidite monomer is added, (iii) a 2-cyanoethyl group that protects the second hydroxyl on the phosphite, and (iv) a variable group (in some instances heterocyclic bases such as N(6)-benzyol, N(2)-isobutyryl, N(4)-benzyol, or N(2)-dimethylformamidyl) that protects the amino group on the nitrogenous base.

The phosphoramidite method comprises an iterative 4 step process where phosphoramidite monomers are added in 3′- to 5′-direction, one per cycle; where the iterative steps comprise a first activation and coupling step, a second capping step, a third oxidation step, and a fourth detritylation step. The first phosphoramidite of the sequence is pre-attached to a solid support by a linker entity and must first undergo detritylation to remove 5′ DMT protecting group from the deoxyribose sugar, generating a free 5′ hydroxyl group. Following detritylation, a first activation and coupling step occurs where an excess of the next appropriate phosphoramidite monomer is added and activated by an activator molecule, where the activator molecule may be tetrazole or any derivative molecule. Activation displaces the diisopropylamino protecting group and couples the next appropriate phosphoramidite monomer through the generation of a new phosphorus-oxygen bond, creating a phosphite triester bond between the first phosphoramidite monomer and the next appropriate phosphoramidite monomer. The first activation and coupling step typically yield approximately 99.5% conversion efficiency.

To account for the remaining 0.5% free 5′ hydroxyl groups, the second capping step comprises an acetylation reaction that rapidly acetylates the remaining 5′ hydroxyl groups. The second capping step insures that during the next coupling step, there are no left over free 5′ hydroxyl groups for the next appropriate phosphoramidite monomer to inadvertently interact with. This prevents the desired oligonucleotide sequence from acquiring deletion mutations and prevents the final sequence product from having a mixture of incorrect oligonucleotide sequences.

Following the second capping step, a third oxidation step comprises converting the unstable phosphite triester bond to a more stable phophotriester bond canonically found in the backbone of DNA and RNA oligos. Conversion of the phosphite triester bond is achieved by iodine oxidation in the presence of water and pyridine, removing the 2-cyanoethyl protection group from the second hydroxyl on the phosphite.

To conclude the final step in the synthesis cycle, a fourth detritylation step occurs, where the DMT protecting group at the 5′ end of the next appropriate phosphoramidite monomer is removed, allowing the cycle to continue until the desired length of the oligonucleotide is synthesized. Moreover, deprotection (e.g., removal of DMT) with trichloroacetic acid in dichloromethane gives off an orange color that can be absorbed by a detector and further used to quantify coupling efficiency during each cycle.

Once the oligonucleotide as reached the desired length (i.e., for aptamers between 20-100 nucleotides) the linker entity attaching 3′end of the oligonucleotide to the solid support is cleaved under specific conditions of concentrated ammonium hydroxide. The cleaved oligonucleotide solution is collected and further undergoes deprotection to remove residual protecting groups from the nitrogen base (a variable group such as heterocyclic bases such as N(6)-benzyol, N(2)-isobutyryl, N(4)-benzyol, or N(2)-dimethylformamidyl) before being purified.

Purified aptamer sequences, once experimentally validated to have one or more characteristics can then be utilized in subsequent in vitro binding selections (e.g., phages display or SELEX) where the given molecular target is exposed to the synthesized aptamers. A separation protocol may be used to remove non-binding aptamers (e.g., flow-through). The binding aptamers may then be eluted from the given target. The binding and/or non-binding aptamers may be sequenced to identify the sequence of aptamers that do and/or those that do not bind the given target. This binding selection process may be repeated for any number of cycles (e.g., 1 to 3 cycles) to validate which of the identified/designed aptamers from block 1215 actually bind the given target. In some instances, the subsequent binding selections are performed using aptamers carrying Unique Molecular Identifiers (UMI) to enable accurate counting of copies of a given candidate sequence in elution or flow-through. Because the sequence diversity is reduced at this stage, there can be more copies of each aptamer to interact with the given target and improve the signal to noise ratio (and label quality).

The processes in blocks 1205-1225 may be performed once or repeated in part or in their entirety any number of times to decrease the absolute number of sequences and increase the signal to noise ratio, which ultimately results in a set of aptamer candidates that satisfy the one or more characteristics (e.g., bind targets of interest in a inhibitory/activating fashion or with a certain binding affinity). As used herein, “satisfy” the one or more characteristics can be complete satisfaction (e.g., bound to the target, achieved the certain level of binding affinity, etc.), substantial satisfaction (e.g., bound to the target with an affinity above/below a given threshold or greater than 98% percent inhibition of a function A), or partial satisfaction (e.g., bound to the target at least 60% of the time or greater than 60% percent inhibition of a function A).

The satisfaction of the one or more characteristics may be measured using one or more binding and/or analytical assays, as described in detail herein with respect to FIG. 2. The output from block 1225 (e.g., bulk validation) may include aptamers that can bind to the target with varying strengths (e.g., high, medium or low affinities). The output from block 1225 may also include aptamers that are not be capable of binding to the target. In some instances, the sequences of binding aptamers, non-binding aptamers, or a combination thereof obtained from block 1225 are used to improve the machine learning models in block 1215 (e.g., by retraining the machine learning algorithms). The sequences of binding aptamers, non-binding aptamers, or a combination thereof from block 1225 may be labeled with one or more sequence properties. As described herein, the one or more sequence properties may include a binding-approximation metric that indicates whether an aptamer included in or associated with the training data bound to a particular target and/or a functional-approximation metric that indicates whether a aptamer included in or associated with the training data functions as intended (e.g., inhibits function A). In certain instances, the binding-approximation metric is determined from the subsequent in vitro binding selections (e.g., phages display or SELEX) performed in block 1225 and/or a low-throughput assay, such as in vitro BLI.

At block 1230, the sequences of binding aptamers, non-binding aptamers, or a combination thereof (labeled with one or more sequence properties and/or experimental results) obtained from block 1225 are used to train a reward model (e.g., fine-tuned reward model 335 as described with respect to FIG. 3) and decoder model (e.g., fine-tuned decoder model 635 as described with respect to FIG. 6) to identify sequences of aptamers that can satisfy the one or more characteristics (e.g., high binding affinity). The decoder model is trained such that it generates sequences which the reward model predicts to satisfy the one or more characteristics. The decoder model generates each sequence by choosing each nucleotide one at a time from left-to-right, conditioning auto-regressively on the preceding nucleotides. As described in detail with respect to FIGS. 1-11B, the training process proceeds algorithmically in a loop comprising the following three steps: a) the decoder model is used to generate a large batch (e.g., greater than 50,000) of sequences based on input data, b) the reward model predicts a function-approximation metric for the one or more constraints or characteristics of an aptamer associated with each of the generate sequences, which essentially scores each of the generated sequences, and c) the parameters of the decoder model are updated via a single optimizer step so as to minimize a loss function. Optimization techniques such as proximal policy optimization, sequence entropy bonus, and/or KL divergence objective functions may be used at this stage to facilitate training of the decoder model to identify the hundreds of additional or alternative sequences of aptamers that can satisfy the one or more constraints (e.g., bind a given target) and/or include one or more characteristics (e.g., high binding affinity).

The trained decoder model may then be deployed to a production environment and used to perform a search process (e.g., genetic search) to identify sequences that have one or more characteristics. The sequences of aptamers identified by the decoder model in block 1230 may then be output, as shown in block 1235. The output may comprise hundreds to thousands of aptamer sequences. These sequences may be novel sequences that could not be discovered using a binding selection experiment.

At block 1240, identified or designed aptamer sequences from block 1235 may be used to synthesize new aptamers as described in detail herein with respect to block 1225. These new aptamers may then be characterized or validated using experiments in block 1240. The experiments may include high throughput binding selections (e.g., SELEX) or low-throughput assays. In some instances, the low-throughput assay (e.g., BLI) is used to validate or measure a binding strength (e.g., affinity, avidity, or dissociation constant) of an aptamer to the given target. In this context, BLI may include preparing a biosensor tip to include the aptamers in an immobilized form and a solution with the given target in a tip of a biosensor. Binding between the molecule(s) and the particular target increases a thickness of the tip of the biosensor. The biosensor is illuminated using white light, and an interference pattern is detected. The interference pattern and temporal changes to the interference pattern (relative to a time at which the molecules and particular target are introduced to each other) are analyzed to predict binding-related characteristics, such as binding affinity, binding specificity, a rate of association, and a rate of dissociation. In other instances, the low-throughput assay (e.g., a spectrophotometer to measure protein concentration) is used to validate or measure functional aspects of the aptamer such as its ability to inhibit a biological function (e.g., protein production).

The processes in blocks 1205-1240 may be performed once or repeated in part or in their entirety any number of times to decrease the absolute number of sequences and increase the signal to noise ratio, which ultimately results in a set of aptamer candidates that best satisfy the one or more constraints (e.g., bind targets of interest in a inhibitory/activating fashion or to deliver a drug/therapeutic to a target such as a T-Cell). The output from block 1240 (e.g., BLI) may include aptamers that can bind to the target with varying strengths (e.g., high, medium, or low affinities). The output from block 1240 may also include aptamers that are not be capable of binding to the target. In some instances, the sequences of binding aptamers, non-binding aptamers, or a combination thereof obtained from block 1240 are used to improve the machine learning models in block 1215 and/or 1230 (e.g., by retraining the machine learning algorithms). The sequences of binding aptamers, non-binding aptamers, or a combination thereof from block 1240 may be labeled with one or more sequence properties. As described herein, the one or more sequence properties may include a binding-approximation metric that indicates whether an aptamer included in or associated with the training data bound to a particular target and/or a functional-approximation metric that indicates whether a aptamer included in or associated with the training data functions as intended (e.g., inhibits function A or binds with an affinity greater than X). In certain instances, the binding-approximation metric is determined from the subsequent in vitro BLI performed in block 1240.

In block 1245, a determination is made as to whether one or more of the aptamers evaluated in block 1240 satisfy the one or more characteristics such as the design criteria proposed for an aptamer, the problem being solved (e.g., finding an aptamer that is capable of binding to target with a high-affinity), and/or the answer to a query (e.g., what aptamers are capable of inhibiting function A). The determination may be made based on the binding-approximation metric and/or the functional-approximation metric associated with an aptamer satisfying the one or more characteristics. In some instances, an aptamer design criterion may be used to select one or more aptamers to be output as the final solution to the given problem. For example, the design criteria in block 1245 may include a binding strength (e.g., a cutoff value), a minimum affinity or avidity between the aptamer and the target, or a maximum dissociation constant.

In block 1250, one or more aptamers from experiments 1240 that are determined to satisfy the one or more constraints (e.g., showing affinity greater or equal to the minimum cutoff) are provided, for example, as the final solution to the given problem or as a result to a given query. The providing the output may include generating or synthesizing an output library comprising the final set of aptamers. The output library may be generated or synthesized incrementally as new aptamers are generated and selected by performing and/or repeating blocks 1205-1245. At each repetition cycle one or more aptamers may be identified (i.e., designed, generated and/or selected) and added to the output based on their ability to satisfy the one or more constraints. The providing the output may further include transmitting the one or more aptamer sequences or output library to a user (e.g., transmitting electronically via wired or wireless communication, or in the instance of physical aptamers, transported via delivery service).

It will be appreciated that although FIG. 12 and the description herein, describe going from trillions of sequences to thousands of sequences to hundreds of sequences, these numbers are merely provided for illustrative purposes. In general, it should be understood that pipeline 1200 is provisioned to start with a large data set (a large absolute number of experimentation sequences which could be, for example, septillions, trillions, billions, or millions) for training a highly-parametrized algorithm and eventually narrows down the absolute number of experimentation sequences to a more manageable number eventually aligning on a small data set (a small absolute number of experimentation sequences which could be, for example, hundreds, tens, or less) for low-throughput characterization and validation as potential therapeutic candidates.

Purified aptamer sequences, once experimentally validated to bind a target with one or more characteristics (e.g., strong affinity and high specificity) can then be applied in therapeutics and drug discovery. Over the past several years, aptamers have started to encroach on small protein molecules and antibody therapeutics as more attractive molecules for drug discovery research. This can be attributed to their potential to overcome two of the major shortcomings in drug discovery: insufficient validation of therapeutic targets and insufficient specificity of drug candidates. Compared to many small protein molecules, aptamers bind to their target in the picomolar to nanomolar range, allowing them to out compete other competitive molecules that may otherwise bind. Similar to antibodies, aptamers are designed to only interact with certain molecules, particular isoforms, or even a specific conformational state of their target protein, making them incredibly specific. On the other hand, aptamers can outperform antibodies due to (i) their smaller size allowing aptamers to access sterically hindered protein regions that antibodies cannot, (ii) their ability to be modified by medicinal chemistry, improving aptamer stability by making them more resistant to nuclease and enzymatic degradation and also improving their overall pharmacokinetic properties, and (iii) aptamers do not illicit an immune response making them more favorable drug therapeutics for allergy or autoimmune diseases.

### Techniques for Aptamer Design by Reinforcement Learning Based Fine-Tuning of Generative Language Models

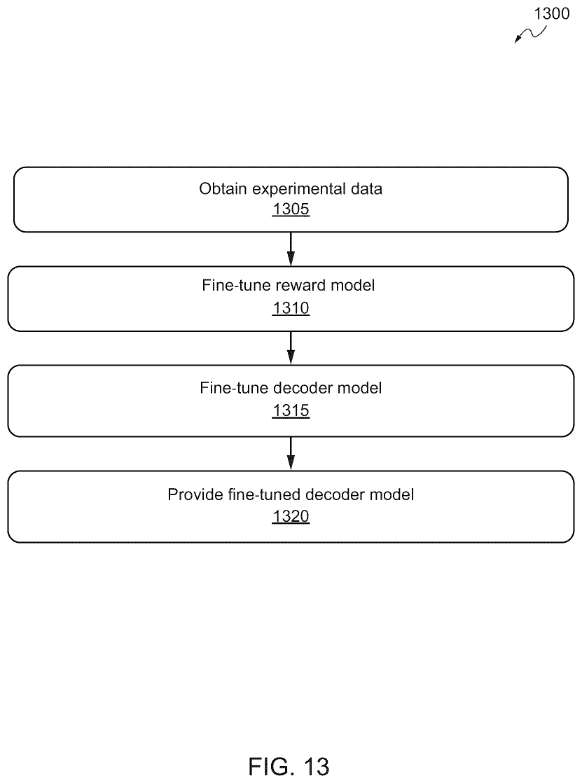

FIG. 13 is a flowchart illustrating process 1300 for training reward and decoder models using a machine learning modeling system (e.g., data collection and modeling system 200 described with respect to FIG. 2). The processing depicted in FIG. 13 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 13 and described below is intended to be illustrative and non-limiting. Although FIG. 13 depicts the various processing steps occurring in a particular sequence or order, this is not intended to be limiting. In certain alternative embodiments, the steps may be performed in some different orders, or some steps may also be performed in parallel.

Process 1300 begins at block 1305, experimental data is obtained, using an experimental assay, for a set of aptamers. The experimental data comprises multiple pairs of data, each pair of data comprising: (i) an aptamer sequence for an aptamer from the set of aptamers, and (ii) a measurement for the characteristic of the aptamer with respect to the given target. In some instances, the experimental assay is a hydrogel particle display plate assay. In certain instances, the characteristic is binding affinity or binding specificity of the aptamer with respect to the given target. The set of aptamers may comprise less than 1000 aptamers.

At block 1310, a reward model is fine-tuned, using the experimental data, to predict the function-approximation metric for the characteristic of each aptamer in the set of aptamers. In some instances, the reward model is an ensemble of a BERT model and an XGBoost decision tree model. The fine-tuning of the reward model may comprise fine-tuning, using the experimental data, both the BERT model and the XGBoost decision tree model to predicted via regression the function-approximation metric for the characteristic of each aptamer in the set of aptamers.

In some instance, the fine-tuning of the BERT model comprises: calculating, using a first reward loss function, a first reward loss for predicting the function-approximation metric for the characteristic of an aptamer, and updating model parameters of the BERT model based on the first reward loss. The first reward loss represents an error between the function-approximation metric and the measurement for the characteristic of the aptamer obtained from the experimental data.

In some instances, the fine-tuning of the XGBoost decision tree model comprises: calculating, using a second reward loss function, a second reward loss for predicting the function-approximation metric for the characteristic of an aptamer, and updating model parameters of the XGBoost decision tree model based on the second reward loss. The second reward loss represents an error between the function-approximation metric and the measurement for the characteristic of the aptamer obtained from the experimental data.

In some instances, prior to the fine-tuning of the reward model, pretraining, using a corpus of ncRNAs and a MLM objective, the BERT model to predict missing nucleotides in an input ncRNA sequence based on context provided by surrounding nucleotides. The pretraining comprises masking some of nucleotides in the input ncRNA sequence to generate the missing nucleotides and training the model to predict the masked nucleotides based on the context of non-masked words.

In some instances, each pair of data further comprises: (i) the aptamer sequence for the aptamer from the set of aptamers, and (ii) a measurement for a characteristic of the aptamer and a different measurement for a different characteristic of the aptamer, and the process 1300 further comprise fine-tuning, using the experimental data, another reward model to predict a different function-approximation metric for the different characteristic of each aptamer in the set of aptamers.

At block 1315, a decoder model is fine-tuned for generating novel aptamer sequences for a set of novel aptamers. Fine-tuning of the decoder model comprises: (i) inputting aptamer sequences for a set of aptamers into a decoder model, (ii) generating, using the decoder model, novel aptamer sequences for a set of novel aptamers predicted to have a measurement at the desirable level for the characteristic of a novel aptamer with respect to the given target, (iii) inputting the novel aptamer sequences into the reward model, (iv) predicting, using the reward model, one or more measurement proxies (reward) for the characteristics of each novel aptamer represented by each of the novel aptamer sequences, and (v) optimizing, using reinforcement learning, model parameters of the decoder model that dictate sampling probabilities of the actions used to generate the novel aptamer sequences. Each aptamer of the set of aptamers has a characteristic with respect to a given target that is measured at a desirable level, the decoder model comprises a function that is configured to return an action given a state, preceding nucleotides in an aptamer sequence are the state, and a nucleotide chosen to come next in the aptamer sequence is the action. The novel aptamer sequences are generated by choosing each nucleotide or action one at a time from left-to-right, conditioning auto-regressively on the state or preceding nucleotides.

The optimizing comprises calculating, using a first loss function, a first loss based on the sampling probabilities and differences in the reward predicted for each of the novel aptamer sequences. The first loss function is configured to optimize sequential decision for choosing each nucleotide or action to maximize the reward. The optimizing further comprises adjusting the model parameters based on the first loss to finetune the decoder model for generating subsequent novel aptamer sequences for a subsequent set of novel aptamers having a more desirable measurement for the characteristic.

In some instances, the optimizing further comprises sampling the actions. The actions have corresponding sampling probabilities and differences in the reward predicted for each of the novel aptamer sequences are measurable and associable to the actions.

In some instances, the optimizing further comprises: calculating, using a second loss function, a second loss based on the sampling probabilities and differences in the reward predicted for each of the novel aptamer sequences, and adjusting the model parameters based on the first loss and the second loss to finetune the decoder model for generating the subsequent novel aptamer sequences for the subsequent set of novel aptamers having a more desirable measurement for the characteristic. The second loss function is configured to compute entropy of the next nucleotide or action prediction distributions output by the decoder model, summed over an entire aptamer sequence.

In some instances, the optimizing further comprises: calculating, using a third loss function, a third loss based on the sampling probabilities and differences in the reward predicted for each of the novel aptamer sequences, and adjusting the model parameters based on the first loss, the second loss, and the third loss to finetune the decoder model for generating the subsequent novel aptamer sequences for the subsequent set of novel aptamers having a more desirable measurement for the characteristic. The third loss function may be configured to apply a penalty for divergence of a next nucleotide or action prediction from a distribution of the decoder model. The first loss function may be a proximal policy optimization objective function, the second loss function may be a sequence entropy bonus objective function, and the third loss function may be a KL divergence objective function.

In some instances, the fine-tuning is performed iteratively until an average reward is equal to or greater than an average reward target threshold. In such an instance, the process 1300 further comprises calculating the average reward using the function-approximation metric predicted for the characteristic of each novel aptamer; when the average reward is less than the average reward target threshold, optimizing, using the reinforcement learning, the model parameters of the decoder model; and when the average reward is equal to or greater than the average reward target threshold, providing the fine-tuned decoder model.

In some instances, the decoder model is a generative language model with a decoder-only transformer architecture. The generative language model may be pretrained using a corpus of short non-coding RNA sequences.

At block 1320, the fine-tuned decoder model is provided. For example, the fine-tuned decoder model may be provided to a machine learning operationalization framework (e.g., sequence or aptamer prediction subsystem 203 and an optional analysis prediction subsystem 204 described with respect to FIG. 2) comprising hardware such as one or more processors (e.g., a CPU, GPU, TPU, FPGA, the like, or any combination thereof), memory, and storage that operates deployment tools including software or computer program instructions (e.g., Application Programming Interfaces (APIs), Cloud Infrastructure, Kubernetes, Docker, TensorFlow, Kuberflow, Torchserve, and the like) to execute arithmetic, logic, input and output commands for executing the fine-tuned decoder model in a production environment. In some instances, the deployment tools implement deployment of the fine-tuned decoder model using a cloud platform such as Amazon Web Services (AWS), Google Cloud Platform (GCP), and Microsoft Azure. A cloud platform makes machine learning more accessible, flexible, and cost-effective while allowing developers to build and deploy the fine-tuned decoder model faster.

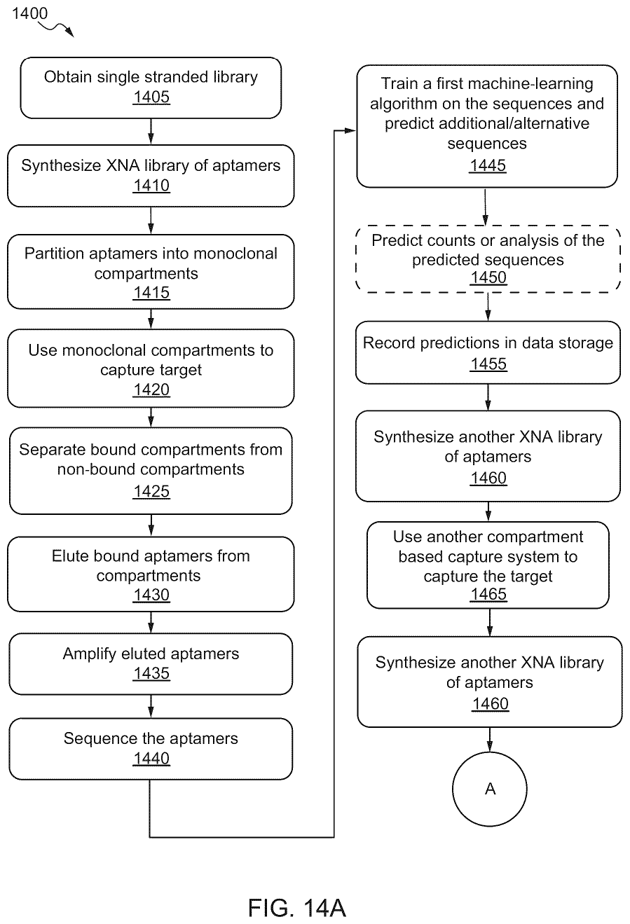

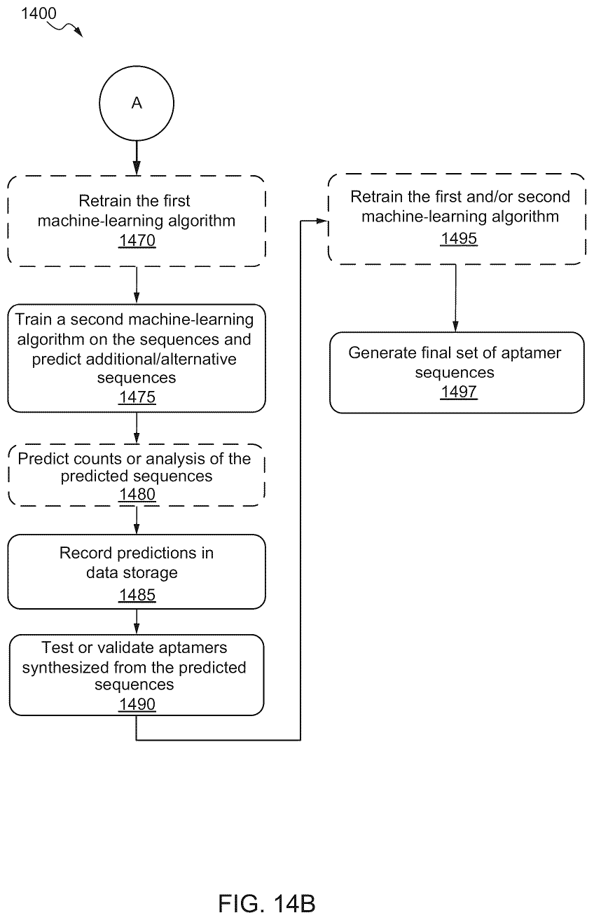

FIGS. 14A and 14B are flowcharts illustrating process 1400 for developing aptamers using a machine learning modeling system and an aptamer development platform (e.g., data collection and modeling system 200 described with respect to FIG. 2). The processing depicted in FIGS. 14A and 14B may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIGS. 14A and 14B and described below is intended to be illustrative and non-limiting. Although FIGS. 14A and 14B depicts the various processing steps occurring in a particular sequence or order, this is not intended to be limiting. In certain alternative embodiments, the steps may be performed in some different orders, or some steps may also be performed in parallel.

Process 1400 begins at block 1405, at which one or more single stranded DNA or RNA (ssDNA or ssRNA) libraries are obtained. The one or more ssDNA or ssRNA libraries comprise a plurality of ssDNA or ssRNA sequences. At block 1410, an XNA aptamer library is synthesized from the one or more ssDNA or ssRNA libraries. The XNA aptamer sequences that make up the XNA aptamer library may be synthesized in vitro with a transcription assay that includes enzymatic or chemical synthesis. The XNA aptamer library comprises a plurality of aptamer sequences. It will be appreciated that techniques disclosed herein can be applied to assess other aptamers rather than XNA aptamers. For example, alternatively or additionally, the techniques described herein may be used to assess the interactions between any type of sequence of nucleic acids (e.g., DNA and RNA) and epitopes of a target. Thus, the following block may synthesize a DNA or RNA aptamer library as input for aptamer sequences rather than constructing an XNA library.

At block 1415, the plurality of aptamers within the XNA aptamer library (optionally DNA or RNA libraries) are partitioned into monoclonal compartments that combined establish a compartment-based capture system. Each monoclonal compartment comprises a unique aptamer from the plurality of aptamers. In some instances, the one or more monoclonal compartments are one or more monoclonal beads. In some instances, each monoclonal compartment or unique aptamer comprises a unique barcode (e.g., a unique molecular identifiers such as a unique sequence of nucleotides) for tracking identification of the compartment and/or the aptamer associated with the monoclonal compartment. At block 1420, the compartment-based capture system is used to capture one or more targets. The capturing comprises the one or more targets binding to the unique aptamer within one or more monoclonal compartments. In some instances, the one or more targets are identified based on a query received from a user. As used herein, when an action is “based on” something, this means the action is based at least in part on at least a part of the something. At block 1425, the one or more monoclonal compartments of the compartment-based capture system that comprise the one or more targets bound to the unique aptamer are separated from a remainder of monoclonal compartments of the compartment-based capture system that do not comprise the one or more targets bound to a unique aptamer. In some instances, the one or more monoclonal compartments are separated from the remainder of monoclonal compartments using a fluorescence-activated cell sorting system.

At block 1430, the unique aptamer is eluted from each of the one or more monoclonal compartments and/or the one or more targets. At block 1435, the unique aptamer from each of the one or more monoclonal compartments is amplified by enzymatic or chemical processes. At block 1440, the unique aptamer from each of the one or more monoclonal compartments (e.g., the bound aptamers) are sequenced. The sequencing comprises using a sequencer (and optionally an additional assay such as BLI or a spectrometer) to generate sequencing data and optionally analysis data (e.g., a binding-approximation metric and/or functional-approximation metric) for the unique aptamer from each of the one or more monoclonal compartments. The analysis data for the unique aptamer from each of the one or more monoclonal compartments may indicate the unique aptamer did bind to the one or more targets. In some instances, the sequencing further comprises generating count data for the unique aptamer from each of the one or more monoclonal compartments. In some instances, the sequencing further comprises sequences of unique aptamers from the remainder of the monoclonal compartments (e.g., non-bound aptamers). The sequencing further comprises using a sequencer (and optionally an additional assay such as BLI or a spectrometer) to generate sequencing data and optionally analysis data (e.g., a binding-approximation metric and/or functional-approximation metric) for the unique aptamer from each of the remainder of the monoclonal compartments (e.g., non-bound aptamers). The analysis data for the unique aptamer from each of the one or more monoclonal compartments may indicate the unique aptamer did not bind to the one or more targets.

At block 1445, the selection sequence data and optionally the count and/or analysis data are used for training a first machine learning algorithm (e.g., a highly parametric machine learning algorithm such as a neural network or ensemble of neural networks) to generate a first trained machine learning model. Thereafter, aptamer sequences are identified, by the first trained machine learning model, as potentially satisfying one or more characteristics, e.g., an initial solution for a given problem such as a high affinity binder to a given target. The identification may comprise inputting a subset of sequences from the selection sequence data (from block 1440), sequences from a pool of sequences different from the sequences from the selection sequence data, or a combination thereof into the first trained machine learning model, estimating, by the first trained machine learning model, a fitness score of each input sequence (the fitness scores is a measure of how well a given sequence performs as a solution with respect to the given problem), and identifying aptamer sequences that satisfy the given problem based on the estimated fitness score for each sequence. In some instances, additional techniques including the application of one or more different types of algorithms such as search algorithms (e.g., a genetic algorithm) or optimization algorithms (e.g., linear optimization) are used in combination with the first trained machine learning model to improve upon the identification of aptamer sequences. For example, the aptamer sequences identified by the first trained machine learning model may be evolved using a genetic algorithm to identify or design aptamer sequences that satisfy the given one or more characteristics, as described in detail herein.

Optionally at block 1450, a count and/or analysis of the identified aptamer sequences is predicted by one or more prediction models. At block 1455, the identified aptamer sequences and optionally the predicted analysis data and/or count data are recorded in a data structure in association with the one or more targets.

At block 1460, another XNA aptamer library (optionally a DNA or RNA library) is synthesized from the identified aptamer sequences. The aptamers within the another XNA aptamer library (optionally a DNA or RNA library) are partitioned into monoclonal compartments that combined establish another compartment-based capture system. Each monoclonal compartment comprises a unique aptamer from the plurality of aptamers. At block 1465, another compartment-based capture system is used to capture the one or more targets. The capturing comprises the one or more targets binding to the unique aptamer sequence within one or more monoclonal compartments. Thereafter, as described similarly with respect to blocks 1425-1440, the one or more monoclonal compartments of the another compartment-based capture system that comprise the one or more targets bound to the unique aptamer are separated from a remainder of monoclonal compartments of the another compartment-based capture system that does not comprise the one or more targets bound to a unique aptamer. The unique aptamer is then eluted from each of the one or more monoclonal compartments and/or the one or more targets, amplified by enzymatic or chemical processes, and sequenced.

The sequencing comprises using a sequencer (and optionally an additional assay such as BLI or a spectrometer) to generate sequencing data and optionally analysis data (e.g., a binding-approximation metric and/or functional-approximation metric) for the unique aptamer from each of the one or more monoclonal compartments. The analysis data for the unique aptamer from each of the one or more monoclonal compartments may indicate the unique aptamer did bind to the one or more targets. In some instances, the sequencing further comprises generating count data for the unique aptamer from each of the one or more monoclonal compartments. In some instances, the sequencing further comprises sequences of unique aptamers from the remainder of the monoclonal compartments (e.g., non-bound aptamers). The sequencing further comprises using a sequencer (and optionally an additional assay such as BLI or a spectrometer) to generate sequencing data and optionally analysis data (e.g., a binding-approximation metric and/or functional-approximation metric) for the unique aptamer from each of the remainder of the monoclonal compartments (e.g., non-bound aptamers). The analysis data for the unique aptamer from each of the one or more monoclonal compartments may indicate the unique aptamer did not bind to the one or more targets.

Optionally at block 1470, the selection sequence data and optionally the count and/or analysis data from block 1465 are used as supplemental training data for retraining the first machine learning algorithm (e.g., a highly parametric machine learning algorithm such as a neural network or ensemble of neural networks) to generate an improved version of the first trained machine learning model. The supplemental training data can have a higher accuracy, and/or can have a higher precision relative the accuracy, and/or precision of the training data. For example, the sequences and corresponding count and analysis data in the original training data used in block 1445 will have more noise and thus a lower signal to noise ratio, while the noise in the supplemental training data will be lower and thus a higher signal to noise ratio. Thereafter, aptamer sequences are identified, by the improved version of the first trained machine learning model, as potentially satisfying one or more characteristics, e.g., an initial solution for a given problem.

At block 1475, some or all of the selection sequence data and optionally the count and/or analysis data (from block 1440), the selection sequence data and optionally the count and/or analysis data (from block 1465), or a combination thereof are used for training a second machine learning algorithm (e.g., a decoder machine learning algorithm as described in process 1300 with respect to FIG. 13) to generate a second trained machine learning model. Thereafter, aptamer sequences are identified, by the second trained machine learning model, as satisfying the one or more characteristics, e.g., a final solution for a given problem. The identification may comprise inputting a subset of sequences from the selection sequence data and optionally the count and/or analysis data (from block 1440), a subset of sequences from the selection sequence data and optionally the count and/or analysis data (from block 1465), sequences and optionally the count and/or analysis data from a pool of sequences different from the sequences from the selection sequence data, or a combination thereof into the second trained machine learning model, estimating, by the second trained machine learning model, a fitness score of each input sequence (the fitness scores is a measure of how well a given sequence performs to potentially satisfy the one or more characteristics), and identifying aptamer sequences that potentially satisfy the one or more characteristics based on the estimated fitness score for each sequence. In some instances, additional techniques including the application of one or more different types of algorithms such as search algorithms (e.g., a genetic algorithm) or optimization algorithms (e.g., linear optimization) are used in combination with the second trained machine learning model to improve upon the identification or design of sequences for derived aptamers. For example, identification, by the second trained machine learning model, of the aptamer sequences may be optimized using an optimization algorithm to identify or design aptamer sequences that potentially satisfy the one or more characteristics.

Optionally at block 1480, a count or analysis of the identified aptamer sequences is predicted by one or more prediction models. At block 1485, the identified aptamer sequences and optionally the predicted analysis data and/or count data are recorded in a data structure in association with the one or more targets.

At block 1490, the aptamer sequences identified in block 1475 may be synthesized and tested for satisfying the one or more characteristics (e.g., binding to or inhibiting the target). The testing may include one or more experimental steps comprising BLI and/or functional assays. The BLI and/or functional assays can generate analytical data for the aptamer and target interactions. The interactions may include binding-approximation metrics such as binding and dissociation metrics and/or functional-approximation metrics such as inhibition and promoter metrics.

Optionally at block 1495, the selection sequence data and optionally the count and/or analysis data from blocks 1475-1490 are used as supplemental training data for retraining the first machine learning algorithm (e.g., a highly parametric machine learning algorithm such as a neural network or ensemble of neural networks) and/or the second machine learning algorithm (e.g., a decoder machine learning algorithm) to generate an improved version of the first trained machine learning model and/or the second trained machine learning model. The supplemental training data can have a higher accuracy, and/or can have a higher precision relative the accuracy, and/or precision of the training data. For example, the sequences and corresponding count and analysis data in the original training data used in block 1445 and/or 1465 will have more noise and thus a lower signal to noise ratio, while the noise in the supplemental training data will be lower and thus a higher signal to noise ratio. Moreover, the binding-approximation metric and/or functional-approximation metric in the training data in block 1445 and/or 1465 may include a binary value (because of use of a high-throughput system), while the binding affinity scores in the supplemental training data may include a categorical or numeric value (because of the BLI and/or functional assays). As another example, the binding-approximation metric and/or functional-approximation metric in the training data can include a categorical value (identifying a category within a first set of categories), while the binding-approximation metric and/or functional-approximation metric in the supplemental training data can include a categorical (identifying a category within a second set of categories, where there are more categories in the second set relative to the first set) or numeric value. As yet another example, the binding-approximation metric and/or functional-approximation metric in the training data can include a numeric value with a first number of significant figures, while the binding-approximation metric and/or functional-approximation metric in the supplemental training data can include a numeric value with more significant figurers than the first number of significant figures. Thereafter, aptamer sequences are identified, by the improved version of the first trained machine learning model and/or second trained machine learning model, as potentially satisfying the one or more characteristics, e.g., an initial solution for a given problem.

At block 1497, the analytical data generated in 1490 may be used to generate or curate a final set of aptamer sequences as satisfying the one or more characteristics, e.g., a final solution to a given problem. In some instances, an output library is generated that comprises the final set of aptamer sequences. The output library may be generated incrementally as new aptamer sequences are generated and selected by repeating all or select blocks of blocks 1405-1495 (e.g., several cycles of 1400 or a portion thereof in a loop or interlaced loops). At each repetition cycle one or more aptamer sequences may be identified (i.e., designed, generated and/or selected) and added to the output based on their ability to satisfy the one or more characteristics.

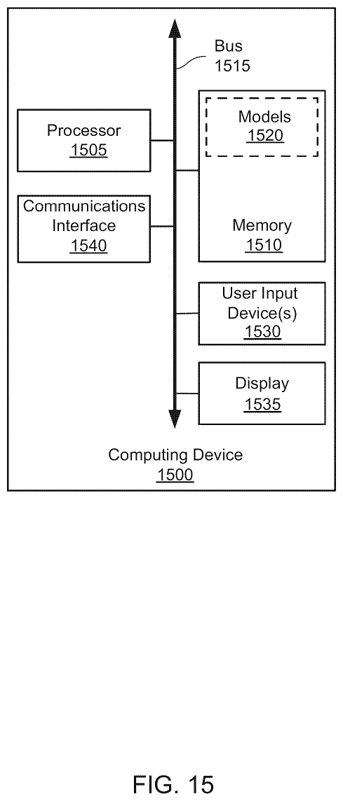

FIG. 15 illustrates an example computing device 1500 suitable for use with systems and methods for developing aptamers and biologics or providing results to a query according to this disclosure. The example computing device 1500 includes a processor 1505 which is in communication with the memory 1510 and other components of the computing device 1500 using one or more communications buses 1515. The processor 1505 is configured to execute processor-executable instructions stored in the memory 1510 to perform one or more methods for developing aptamers or biologics or providing results to a query according to different examples, such as part or all of the example method 1300 or 1400 described above with respect to FIGS. 13 and 14. In this example, the memory 1510 stores processor-executable instructions that provide for provisioning of machine learning algorithms or models 1520, as discussed above with respect to FIGS. 1-12 (e.g., the machine learning operationalization framework).

The computing device 1500, in this example, also includes one or more user input devices 1530, such as a keyboard, mouse, touchscreen, microphone, etc., to accept user input. The computing device 1500 also includes a display 1535 to provide visual output to a user such as a user interface or display of aptamer sequences. The computing device 1500 also includes a communications interface 1540. In some examples, the communications interface 1540 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

### ADDITIONAL CONSIDERATIONS

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A computer-implemented method comprising:
fine-tuning a decoder model for generating novel aptamer sequences for a set of novel aptamers, wherein fine-tuning of the decoder model comprises:
inputting aptamer sequences for a set of aptamers into a decoder model, wherein:
each aptamer of the set of aptamers has a characteristic with respect to a given target that is measured at a desirable level,
the decoder model comprises a function that is configured to return an action given a state,
preceding nucleotides in an aptamer sequence are the state, and
a nucleotide chosen to come next in the aptamer sequence is the action;
generating, using the decoder model, novel aptamer sequences for a set of novel aptamers predicted to have a measurement at the desirable level for the characteristic of a novel aptamer with respect to the given target, wherein the novel aptamer sequences are generated by choosing each nucleotide or action one at a time from left-to-right, conditioning auto-regressively on the state or preceding nucleotides;
inputting the novel aptamer sequences into a reward model;
predicting, using the reward model, one or more measurement proxies for the characteristics of each novel aptamer represented by each of the novel aptamer sequences, wherein the one or more measurement proxies is the reward;
optimizing, using reinforcement learning, model parameters of the decoder model that dictate sampling probabilities of the actions used to generate the novel aptamer sequences, wherein the optimizing comprises;
calculating, using a first loss function, a first loss based on the sampling probabilities and differences in the reward predicted for each of the novel aptamer sequences, wherein the first loss function is configured to optimize sequential decision for choosing each nucleotide or action to maximize the reward; and
adjusting the model parameters based on the first loss to finetune the decoder model for generating subsequent novel aptamer sequences for a subsequent set of novel aptamers having a more desirable measurement for the characteristic; and
providing the fine-tuned decoder model.

2. The computer-implemented method of claim 1, wherein the fine-tuning is performed iteratively until an average reward is equal to or greater than an average reward target threshold.

3. The computer-implemented method of claim 2, further comprising:
calculating the average reward using the function-approximation metric predicted for the characteristic of each novel aptamer;
when the average reward is less than the average reward target threshold, optimizing, using the reinforcement learning, the model parameters of the decoder model; and
when the average reward is equal to or greater than the average reward target threshold, providing the fine-tuned decoder model.

4. The computer-implemented method of claim 1, wherein the decoder model is a generative language model with a decoder-only transformer architecture.

5. The computer implemented method of claim 4, wherein the generative language model is pretrained using a corpus of short non-coding RNA sequences.

6. The computer-implemented method of claim 1, wherein the optimizing further comprises sampling the actions, wherein the actions have corresponding sampling probabilities and differences in the reward predicted for each of the novel aptamer sequences are measurable and associable to the actions.

7. The computer-implemented method of claim 6, wherein the optimizing further comprises:
calculating, using a second loss function, a second loss based on the sampling probabilities and differences in the reward predicted for each of the novel aptamer sequences, wherein the second loss function is configured to compute entropy of the next nucleotide or action prediction distributions output by the decoder model, summed over an entire aptamer sequence; and
adjusting the model parameters based on the first loss and the second loss to finetune the decoder model for generating the subsequent novel aptamer sequences for the subsequent set of novel aptamers having a more desirable measurement for the characteristic.

8. The computer-implemented method of claim 7, wherein the optimizing further comprises:
calculating, using a third loss function, a third loss based on the sampling probabilities and differences in the reward predicted for each of the novel aptamer sequences, wherein the third loss function is configured to apply a penalty for divergence of a next nucleotide or action prediction from a distribution of the decoder model; and
adjusting the model parameters based on the first loss, the second loss, and the third loss to finetune the decoder model for generating the subsequent novel aptamer sequences for the subsequent set of novel aptamers having a more desirable measurement for the characteristic.

9. The computer-implemented method of claim 8, wherein the first loss function is a proximal policy optimization objective function, the second loss function is a sequence entropy bonus objective function, and the third loss function is a Kullback-Leibner (KL) divergence objective function.

10. The computer-implemented method of claim 1, further comprising:
obtaining, using an experimental assay, experimental data for a set of aptamers, wherein the experimental data comprises multiple pairs of data, each pair of data comprising: (i) an aptamer sequence for an aptamer from the set of aptamers, and (ii) a measurement for the characteristic of the aptamer with respect to the given target; and
fine-tuning, using the experimental data, the reward model to predict the function-approximation metric for the characteristic of each aptamer in the set of aptamers.

11. The computer-implemented method of claim 10, wherein the experimental assay is a hydrogel particle display plate assay.

12. The computer-implemented method of claim 10, wherein the characteristic is binding affinity or binding specificity of the aptamer with respect to the given target.

13. The computer-implemented method of claim 10, wherein the set of aptamers comprises less than 1000 aptamers.

14. The computer-implemented method of claim 10, wherein the reward model is an ensemble of a Bidirectional Encoder Representations from Transformers (BERT) model and an Extreme Gradient Boosted (XGBoost) decision tree model.

15. The computer-implemented method of claim 14, wherein the fine-tuning of the reward model comprises fine-tuning, using the experimental data, both the BERT model and the XGBoost decision tree model to predicted via regression the function-approximation metric for the characteristic of each aptamer in the set of aptamers.

16. The computer-implemented method of claim 15, wherein the fine-tuning of the BERT model comprises:
calculating, using a first reward loss function, a first reward loss for predicting the function-approximation metric for the characteristic of an aptamer, wherein the first reward loss represents an error between the function-approximation metric and the measurement for the characteristic of the aptamer obtained from the experimental data; and
updating model parameters of the BERT model based on the first reward loss.

17. The computer-implemented method of claim 16, wherein the fine-tuning of the XGBoost decision tree model comprises:
calculating, using a second reward loss function, a second reward loss for predicting the function-approximation metric for the characteristic of an aptamer, wherein the second reward loss represents an error between the function-approximation metric and the measurement for the characteristic of the aptamer obtained from the experimental data; and
updating model parameters of the XGBoost decision tree model based on the second reward loss.

18. The computer-implemented method of claim 15, further comprising, prior to the fine-tuning of the reward model, pretraining, using a corpus of non-coding RNA sequences (ncRNAs) and a Masked Language Model (MLM) objective, the BERT model to predict missing nucleotides in an input ncRNA sequence based on context provided by surrounding nucleotides, wherein the pretraining comprises masking some of nucleotides in the input ncRNA sequence to generate the missing nucleotides and training the model to predict the masked nucleotides based on the context of non-masked words.

19. The computer-implemented method of claim 10, wherein each pair of data further comprises: (i) the aptamer sequence for the aptamer from the set of aptamers, and (ii) a measurement for a characteristic of the aptamer and a different measurement for a different characteristic of the aptamer, and the method further comprise fine-tuning, using the experimental data, another reward model to predict a different function-approximation metric for the different characteristic of each aptamer in the set of aptamers.
